(19) United States
(12) Patent Application Publication
(10) Pub. No.: US 2021/0193267 A1
(43) Pub. Date: Jun. 24, 2021

(54) METHODS, SYSTEMS, AND RELATED COMPUTER PROGRAM PRODUCTS FOR EVALUATING CANCER MODEL FIDELITY

(71) Applicant: THE JOHNS HOPKINS UNIVERSITY, Baltimore, MD (US)

(72) Inventors: Patrick Cahan, Baltimore, MD (US); Da Peng, Baltimore, MD (US); Rachel Gleyzer, Baltimore, MD (US)

(21) Appl. No.: 17/123,591

(22) Filed: Dec. 16, 2020

Related U.S. Application Data
(60) Provisional application No. 62/949,295, filed on Dec. 17, 2019.

(51) Int. Cl. G16B 40/00; G16B 5/20

(52) U.S. Cl. CPC G16B 40/00; G16B 5/20

(57) ABSTRACT

Provided herein are methods of generating training classifiers and/or evaluating cancer models. Related systems and computer program products are also provided.

### CROSS-REFERENCE TO RELATED APPLICATIONS

This application claims priority to, and the benefit of, U.S. Provisional Patent Application No. 62/949,295 entitled “METHODS, SYSTEMS, AND RELATED COMPUTER PROGRAM PRODUCTS FOR EVALUATING CANCER MODEL FIDELITY” filed Dec. 17, 2019, the disclosure of which is hereby incorporated by reference in its entirety.

### STATEMENT REGARDING FEDERALLY SPONSORED RESEARCH OR DEVELOPMENT

This invention was made with government support under grant number CA228991 awarded by the National Institutes of Health. The government has certain rights in the invention.

### BACKGROUND

Models are widely used to investigate cancer biology and to identify potential therapeutics. Popular modeling modalities are cancer cell lines (CCLs), genetically engineered mouse models (GEMMs), and patient derived xenografts (PDXs). These classes of models differ in the types of questions that they are designed to address. CCLs are often used to address cell intrinsic mechanistic questions, GEMMs to chart progression of molecularly defined-disease, and PDXs to explore patient-specific response to therapy in a physiologically relevant context. Models also differ in the extent to which they represent specific aspects of a cancer type. Even with this intra- and inter-class model variation, all models should represent the tumor type or sub-type under investigation, and not another type of tumor, and not a non-cancerous tissue. Therefore, cancer-models should be selected not only based on the specific biological question but also based on the similarity of the model to the cancer type under investigation (Mouradov et al. (2014) “Colorectal cancer cell lines are representative models of the main molecular subtypes of primary cancer,” Cancer Research, 74(12):3238-3247; Stuckelberger et al. (2018) “Precious GEMMs: emergence of faithful models for ovarian cancer research,” The Journal of Pathology, 245(2):129-131).

Various methods have been proposed to determine the similarity of cancer models to their intended subjects. Domcke et al. devised a ‘suitability score’ as a metric of the molecular similarity of CCLs to high grade serous ovarian carcinoma based on a heuristic weighting of copy number alterations, mutation status of several genes that distinguish ovarian cancer subtypes, and hypermutation status (Domcke et al. (2013) “Evaluating cell lines as tumour models by comparison of genomic profiles,” Nature Communications, 4:2126). Other studies have taken analogous approaches by either focusing on transcriptomic or ensemble molecular profiles (e.g. transcriptomic and copy number alterations) to quantify the similarity of cell lines to tumors (Jiang et al. (2016) “Comprehensive comparison of molecular portraits between cell lines and tumors in breast cancer,” BMC Genomics 17 Suppl 7:525; Chen (2015) “Relating hepatocellular carcinoma tumor samples and cell lines using gene expression data in translational research,” BMC Medical Genomics 8 Suppl 2:S5.; Vincent et al. (2015) “Assessing breast cancer cell lines as tumour models by comparison of mRNA expression profiles,” Breast Cancer Research 17:114). These studies were tumor-type specific, focusing on CCLs that model, for example, hepatocellular carcinoma or breast cancer. More recently, Yu et al. compared the transcriptomes of CCLs to The Cancer Genome Atlas (TCGA) by correlation analysis, resulting in a panel of CCLs recommended as most representative of 22 tumor types (Yu et al. (2019) “Comprehensive transcriptomic analysis of cell lines as models of primary tumors across 22 tumor types,” Nature Communications 10(1):3574). While all of these studies have provided valuable information, they leave at least two major challenges unmet. The first challenge is to determine the fidelity of GEMMs and PDXs and whether there are stark differences between these classes of models and CCLs. The other major unmet challenge is to allow for rapid assessment of new, emerging cancer models. This challenge is especially relevant now as technical barriers to model generation have been substantially lowered, and because each PDX can be considered a distinct entity requiring validation.

### SUMMARY

The present disclosure relates, in certain aspects, to a computational software tool, called CancerCellNet (CCN), which can be used for several purposes in the clinical and research settings of cancer. A function of the tool is to classify biological samples according to their similarity to over two dozen well-defined cancer tumor types (e.g. breast invasive carcinoma), and sub-types thereof (e.g. ‘luminal A’). This tool is especially useful in cases where the tumor type is difficult for pathologists to determine, such as when the cancer has metastasized and the origin of the primary tumor is unknown. The tool is also useful as a means to gauge the similarity of cancers models to naturally occurring disease. Researchers will be able to use CancerCellNet to determine the model that is most appropriate for their research or translational question.

CancerCellNet uses various types of data, including gene expression or transcriptomic data in certain applications. In some embodiments, the software uses the Random Forest machine learning classification technique. In certain of these embodiments, the training data used to train the algorithm are derived from The Cancer Genome Atlas (TCGA) and/or other data sources. As described herein, CancerCellNet's performance has been assessed on both held out TCGA data, as well as a host of well-annotated tumor data from other sources. The methods and related aspects of the present disclosure also provide a way to transform the data that enables CancerCellNet to be ‘agnostic’ with regards to the type of transcriptomic or other data types. Therefore, the methods are not limited to either microarray data, or RNA-Seq data. In addition, the present disclosure also provides a means of quickly identifying relevant features, which shortens the classifier training time, and makes classification rapid.

In certain aspects, the present disclosure provides a method of generating a training classifier at least partially using a computer. The method includes generating, by the computer, one or more training data sets, wherein a given training data set comprises gene expression profiles of subjects having a given tumor type. The method also includes identifying, by the computer, intersecting genes between the training data sets and one or more query samples to produce one or more intersecting gene sets, and partitioning, by the computer, the intersecting gene sets into training subsets and validation subsets for a given tumor type. The method also includes identifying, by the computer, one or more groups of differentially over-expressed genes, differentially under-expressed genes, and/or least differentially expressed genes in the training subsets to produce one or more baseline gene sets, and generating, by the computer, one or more gene-pairs for one or more of the tumor types from the baseline gene sets. The method also includes pair-transforming, by the computer, the gene-pairs to produce one or more binarized training data sets, and selecting, by the computer, one or more discriminatory gene-pairs for at least some of the tumor types. In addition, the method also includes generating, by the computer, one or more random gene-pair profiles through random permutations of the training data sets, which gene-pair profiles lack tumor type annotation, and selecting, by the computer, one or more of the gene-pairs as features to produce a random forest classifier, thereby generating the training classifier.

In other aspects, the present disclosure provides a method of evaluating a cancer model at least partially using a computer. The method includes generating, by the computer, one or more training data sets, wherein a given training data set comprises gene expression profiles of subjects having a given tumor type, and identifying, by the computer, intersecting genes between the training data sets and one or more query samples to produce one or more intersecting gene sets. The method also includes partitioning, by the computer, the intersecting gene sets into training subsets and validation subsets for a given tumor type, and identifying, by the computer, one or more groups of differentially over-expressed genes, differentially under-expressed genes, and/or least differentially expressed genes in the training subsets to produce one or more baseline gene sets. The method also includes generating, by the computer, one or more gene-pairs for one or more of the tumor types from the baseline gene sets, and pair-transforming, by the computer, the gene-pairs to produce one or more binarized training data sets. The method also includes selecting, by the computer, one or more discriminatory gene-pairs for at least some of the tumor types, and generating, by the computer, one or more random gene-pair profiles through random permutations of the training data sets, which gene-pair profiles lack tumor type annotation. In addition, the method also includes selecting, by the computer, one or more of the gene-pairs as features to produce a random forest classifier, and evaluating one or more cancer models using the random forest classifier.

In some embodiments of the methods, the query samples comprise cancer cell line (CCL) samples, patient derived xenograft (PDX) samples, and/or genetically engineered mouse model (GEMM) samples, or data derived from such sample types. In certain embodiments, the partitioning step comprises randomly sampling the gene expression profiles for the given tumor type. In some embodiments, the methods include down-sampling, up-sampling, and/or log transforming one or more of the training subsets. In certain embodiments, the methods include using log transformed down-sampled counts to produce the baseline gene sets. In some embodiments, the methods include stratifying sampling when selecting gene-pairs as features to produce the random forest classifier. In certain embodiments, the methods include validating the training classifier using the validation subsets. In some embodiments, the methods include pair-transforming the validation subsets.

In some embodiments, the methods include evaluating performance of the training classifier using precision-recall curve and area under the precision-recall curve (AUPR). In certain embodiments, the methods include repeating one or more steps of generating the training classifier. In some embodiments, the methods include using gene-pairs selected from genes listed in Table 1. In certain embodiments, the methods include adding one or more additional features to produce the random forest classifier. In some embodiments, the methods include evaluating one or more cancer cell line (CCL) expression profiles, patient derived xenograft (PDX) expression profiles, and/or genetically engineered mouse model (GEMM) expression profiles using the training classifier. In some embodiments of the methods, the gene-pairs comprise genes from different species.

In certain embodiments of the methods, gene expression profiles comprise RNA-seq and/or microarray gene expression profiles. In some embodiments, the methods also include generating one or more tumor sub-type classifiers. In certain embodiments, the tumor sub-type classifiers comprise one or more gene pairs selected from genes listed in Tables 2-12.

In other aspects, the present disclosure provides a system, comprising a controller comprising, or capable of accessing, computer readable media comprising non-transitory computer executable instruction which, when executed by at least electronic processor perform at least: generating one or more training data sets, wherein a given training data set comprises gene expression profiles of subjects having a given tumor type, and identifying intersecting genes between the training data sets and one or more query samples to produce one or more intersecting gene sets. The electronic processor also performs partitioning the intersecting gene sets into training subsets and validation subsets for a given tumor type, and identifying one or more groups of differentially over-expressed genes, differentially under-expressed genes, and/or least differentially expressed genes in the training subsets to produce one or more baseline gene sets. The electronic processor also performs generating one or more gene-pairs for one or more of the tumor types from the baseline gene sets, and pair-transforming the gene-pairs to produce one or more binarized training data sets. The electronic processor also performs selecting one or more discriminatory gene-pairs for at least some of the tumor types, and generating one or more random gene-pair profiles through random permutations of the training data sets, which gene-pair profiles lack tumor type annotation. In addition, the electronic processor also performs selecting one or more of the gene-pairs as features to produce a random forest classifier, thereby generating the training classifier.

In other aspects, the present disclosure also provides a computer readable media comprising non-transitory computer executable instruction which, when executed by at least electronic processor perform at least: generating one or more training data sets, wherein a given training data set comprises gene expression profiles of subjects having a given tumor type, and identifying intersecting genes between the training data sets and one or more query samples to produce one or more intersecting gene sets. The electronic processor also performs partitioning the intersecting gene sets into training subsets and validation subsets for a given tumor type, and identifying one or more groups of differentially over-expressed genes, differentially under-expressed genes, and/or least differentially expressed genes in the training subsets to produce one or more baseline gene sets. The electronic processor also performs generating one or more gene-pairs for one or more of the tumor types from the baseline gene sets, and pair-transforming the gene-pairs to produce one or more binarized training data sets. The electronic processor also performs selecting one or more discriminatory gene-pairs for at least some of the tumor types, and generating one or more random gene-pair profiles through random permutations of the training data sets, which gene-pair profiles lack tumor type annotation. In addition, the electronic processor also performs selecting one or more of the gene-pairs as features to produce a random forest classifier, thereby generating the training classifier.

In some embodiments of the systems or computer readable media, the query samples comprise cancer cell line (CCL) samples, patient derived xenograft (PDX) samples, and/or genetically engineered mouse model (GEMM) samples. In certain embodiments of the systems or computer readable media, the partitioning step comprises randomly sampling the gene expression profiles for the given tumor type. In some embodiments, the systems or computer readable media include down-sampling, up-sampling, and/or log transforming one or more of the training subsets. In some embodiments, the systems or computer readable media include using log transformed down-sampled counts to produce the baseline gene sets. In some embodiments, the systems or computer readable media include stratifying sampling when selecting gene-pairs as features to produce the random forest classifier. In some embodiments, the systems or computer readable media include validating the training classifier using the validation subsets. In some embodiments, the systems or computer readable media include pair-transforming the validation subsets. In some embodiments, the systems or computer readable media include evaluating performance of the training classifier using precision-recall curve and area under the precision-recall curve (AUPR). In some embodiments, the systems or computer readable media include repeating one or more steps of generating the training classifier.

In some embodiments of the systems or computer readable media, the gene-pairs are selected from genes listed in Table 1. In some embodiments, the systems or computer readable media include adding one or more additional features to produce the random forest classifier. In some embodiments, the systems or computer readable media include evaluating one or more cancer cell line (CCL) expression profiles, patient derived xenograft (PDX) expression profiles, and/or genetically engineered mouse model (GEMM) expression profiles using the training classifier. In some embodiments of the systems or computer readable media, the gene-pairs comprise genes from different species. In some embodiments of the systems or computer readable media, the gene expression profiles comprise RNA-seq and/or microarray gene expression profiles. In some embodiments, the systems or computer readable media further include generating one or more tumor sub-type classifiers. In some embodiments of the systems or computer readable media, the tumor sub-type classifiers comprise one or more gene pairs selected from genes listed in Tables 2-12.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps according to some aspects disclosed herein.

FIG. 2 is a schematic diagram of an exemplary system suitable for use with certain aspects disclosed herein.

FIG. 3A schematically depicts exemplary method steps according to some aspects disclosed herein.

FIG. 3B is a plot of mean area under the precision-recall curve (AUPR) (y-axis) for various cancer types (x-axis).

FIG. 4A are plots showing the performance of a classifier according to certain embodiments disclosed herein for various cancer types in which precision is represented on the y-axis, while recall is represented on the x-axis.

FIG. 4B is a plot of AUPR (y-axis) for various cancer types (x-axis).

FIG. 4C is a plot of AUPR of Cross-Species Testing Data with AUPR represented on the y-axis for various cell types represented on the x-axis.

FIG. 4D schematically depicts exemplary method steps according to some aspects disclosed herein.

FIG. 4E is a plot of cancer subtypes (y-axis) versus mean AUPR (x-axis).

FIG. 5A is a plot of RNA-seq expression data of 657 different cell lines mined across 20 cancer types.

FIG. 5B is a plot of CCN profiles.

FIG. 5C is a plot of classifications.

FIG. 5D is a plot of sub-type classification of Lung Squamous Cell Carcinoma (LUSC) cell lines.

FIG. 5E is a plot of sub-type classification of Lung Adenocarcinoma (LUAD) cell lines.

FIG. 5F is a plot of normalized citation count (y-axis) versus general classification score (x-axis).

FIG. 6A is a plot of AUPR of Microarray Testing Data with AUPR represented on the y-axis for various cancer types represented on the x-axis.

FIG. 6B is a plot of microarray expression data for cancer cell lines mined across various cancer types.

FIG. 6C are plots comparing CCLE classification scores between microarray (y-axis) and RNA-seq data (x-axis).

FIG. 7A is a plot of expression data mined across various cancer types.

FIG. 7B is a plot of CCN profiles.

FIG. 7C is a plot of classifications.

FIG. 7D is a plot of classifications.

FIG. 7E is a plot of classifications.

FIG. 8A is a plot of expression data mined across various cancer types.

FIG. 8B is a plot of CCN profiles.

FIG. 8C is a plot of classifications.

FIG. 8D is a plot of classifications.

FIG. 9 is a plot of classifications.

FIG. 10 are plots of general CCN scores of cancer models compared on a per tumor type basis.

FIG. 11 are plots of sub-type classifications.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Cancer Type: As used herein, “cancer type” or “tumor type” refers to type or subtype of cancer defined, e.g., by histopathology. Cancer type can be defined by any conventional criterion, such as on the basis of occurrence in a given tissue (e.g., blood cancers, CNS, brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestine cancers, soft tissue cancers, thyroid cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Classifier: As used herein, “classifier,” generally refers to algorithm computer code that receives, as input, test data and produces, as output, a classification of the input data as belonging to one or another class.

Machine Learning Algorithm: As used herein, “machine learning algorithm,” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART—classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Sample: As used herein, “sample” means anything capable of being analyzed by the methods and/or systems disclosed herein.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a cancer, is going to receive a cancer therapy, and/or has received at least one cancer therapy. The subject can be in remission of a cancer.

### DETAILED DESCRIPTION

Cancer researchers use, for example, cell lines, patient derived xenografts, and genetically engineered mice as models to investigate tumor biology and to identify therapeutics. The generalizability and power of a model derives from the fidelity with which it represents the tumor type of investigation, however, the extent to which this is true is often unclear. The preponderance of models and the ability to readily generate new ones has created a demand for tools that can measure the extent and ways in which cancer models resemble or diverge from native tumors. In certain aspects, the present disclosure relates to a computational tool, called CancerCellNet (CCN), which measures the similarity of cancer models, in some embodiments, to 25 naturally occurring tumor types and 46 sub-types, in a platform and species agnostic manner. As illustrated in the Examples provided herein, this tool was applied to 657 cancer cell lines, 415 patient derived xenografts, and 26 distinct genetically engineered mouse models, documenting the most faithful models, identifying cancers underserved by adequate models, and finding models with annotations that do not match their classification. By comparing models across modalities, the illustrative Examples further show that genetically engineered mice have higher transcriptional fidelity than patient derived xenografts and cell lines in four out of five tumor types.

Exemplary Methods

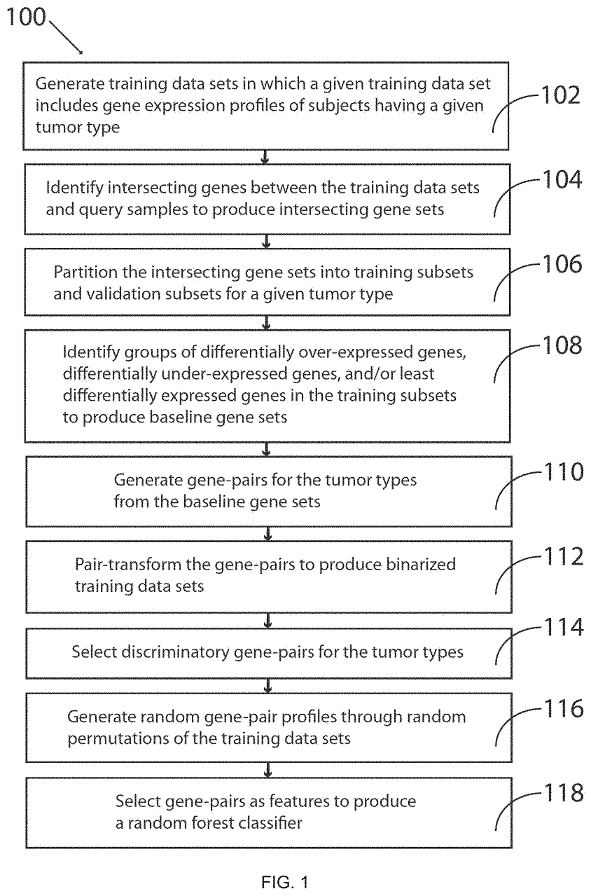

The present disclosure provides various methods of generating training classifiers and/or evaluating cancer models. To illustrate, FIG. 1 is flow chart that schematically depicts exemplary method steps according to some aspects disclosed herein. As shown, method 100 includes generating training data sets in which a given training data set includes gene expression profiles of subjects having a given tumor type (step 102). Typically, one or more of the steps of method 100 are computer implemented. Exemplary systems and computers are described further herein. Method 100 also includes identifying intersecting genes between the training data sets and query samples to produce intersecting gene sets (step 104), and partitioning the intersecting gene sets into training subsets and validation subsets for a given tumor type (step 106). Method 100 also includes identifying groups of differentially over-expressed genes, differentially under-expressed genes, and/or least differentially expressed genes in the training subsets to produce baseline gene sets (step 108), and generating gene-pairs for the tumor types from the baseline gene sets (step 110). Method 100 also includes pair-transforming the gene-pairs to produce binarized training data sets (step 112), and selecting discriminatory gene-pairs for at least some of the tumor types (step 114). In addition, method 100 also includes generating random gene-pair profiles through random permutations of the training data sets (step 116). Typically, these gene-pair profiles lack tumor type annotation. Method 100 also includes selecting gene-pairs as features to produce a random forest classifier to generate the training classifier (step 118). Typically, the methods disclosed herein include evaluating cancer models using the random forest classifier using the training classifier generated by method 100. Aspects of the methods are described further herein, including in the Example.

In some embodiments of the methods, the query samples comprise cancer cell line (CCL) samples, patient derived xenograft (PDX) samples, and/or genetically engineered mouse model (GEMM) samples. In certain embodiments, the partitioning step comprises randomly sampling the gene expression profiles for the given tumor type. In some embodiments, the methods include down-sampling, up-sampling, and/or log transforming one or more of the training subsets. In certain embodiments, the methods include using log transformed down-sampled counts to produce the baseline gene sets. In some embodiments, the methods include stratifying sampling when selecting gene-pairs as features to produce the random forest classifier. In certain embodiments, the methods include validating the training classifier using the validation subsets. In some embodiments, the methods include pair-transforming the validation subsets.

In some embodiments, the methods include evaluating performance of the training classifier using precision-recall curve and area under the precision-recall curve (AUPR). In certain embodiments, the methods include repeating one or more steps of generating the training classifier. In some embodiments, the methods include the gene-pairs are selected from genes listed in Table 1. In certain embodiments, the methods include adding one or more additional features to produce the random forest classifier. In some embodiments, the methods include evaluating one or more cancer cell line (CCL) expression profiles, patient derived xenograft (PDX) expression profiles, and/or genetically engineered mouse model (GEMM) expression profiles using the training classifier. In some embodiments of the methods, the gene-pairs comprise genes from different species.

In certain embodiments of the methods, gene expression profiles comprise RNA-seq and/or microarray gene expression profiles. In some embodiments, the methods also include generating one or more tumor sub-type classifiers. In certain embodiments, the tumor sub-type classifiers comprise one or more gene pairs selected from genes listed in Tables 2-12.

Exemplary Systems and Computer Readable Media

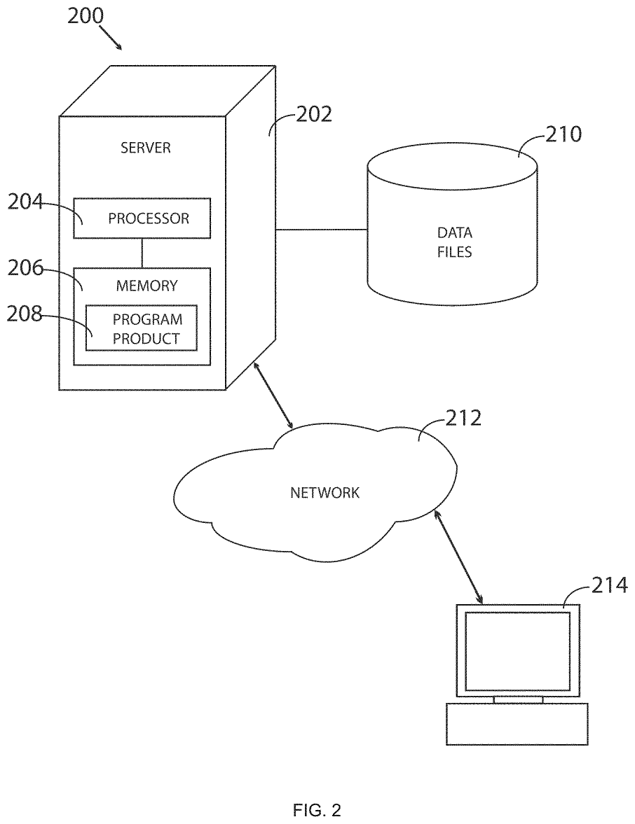

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214 (e.g., client-side computer terminals, telephones, tablets, laptops, other mobile devices, etc.) positioned remote from and in communication with the remote server 202, through electronic communication network 212, such as the Internet or other internetwork. Communication device 214 typically includes an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., control sample or comparator result data, indexed customized therapies, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication device 214 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one CCN model or component thereof, and/or the like to be displayed (e.g., via communication device 214 or the like) and/or receive information from other system components and/or from a system user (e.g., via communication device 214 or the like).

In some aspects, program product 208 includes non-transitory computer-executable instructions which, when executed by electronic processor 204 perform at least: generating one or more training data sets, wherein a given training data set comprises gene expression profiles of subjects having a given tumor type; identifying intersecting genes between the training data sets and one or more query samples to produce one or more intersecting gene sets; partitioning the intersecting gene sets into training subsets and validation subsets for a given tumor type; identifying one or more groups of differentially over-expressed genes, differentially under-expressed genes, and/or least differentially expressed genes in the training subsets to produce one or more baseline gene sets; generating one or more gene-pairs for one or more of the tumor types from the baseline gene sets; pair-transforming the gene-pairs to produce one or more binarized training data sets; selecting one or more discriminatory gene-pairs for at least some of the tumor types; generating one or more random gene-pair profiles through random permutations of the training data sets, which gene-pair profiles lack tumor type annotation; and selecting one or more of the gene-pairs as features to produce a random forest classifier, thereby generating the training classifier.

System 200 also typically includes additional system components that are configured to perform various aspects of the methods described herein. In some of these aspects, one or more of these additional system components are positioned remote from and in communication with the remote server 202 through electronic communication network 212, whereas in other aspects, one or more of these additional system components are positioned local, and in communication with server 202 (i.e., in the absence of electronic communication network 212) or directly with, for example, desktop computer 214.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### Example

This example presents various exemplary aspects of CancerCellNet (CCN). Details of CCN are also described in Peng et al. “Evaluating the transcriptional fidelity of cancer models.” bioRxiv (2020) (10.1101/2020.03.27.012757), the entire disclosure of which, including all supplemental material, is incorporated by reference in its entirety.

Training Broad CancerCellNet

To generate training data sets, 9288 patient tumor non-normalized RNA-seq expression profiles and their corresponding sample tables annotating each patient profile to a cancer type across 25 different tumor types were downloaded from TCGA using TCGAWorkflowData, TCGAbiolinks (Silva et al. (2016) “TCGA Workflow: Analyze cancer genomics and epigenomics data using Bioconductor packages,” [version 2; peer review: 1 approved, 2 approved with reservations]. F1000Research 5:1542) and SummarizedExperiment (Morgan et al. (2018) SummarizedExperiment: SummarizedExperiment container) packages. After compiling the patient tumor dataset, the intersecting genes between TCGA dataset and all the query samples (CCLs, PDXs, GEMMs) were found, and only those genes were used as features for building the classifier. Two-thirds of the patient tumor profiles from each cancer category randomly sampled as the training set and the rest were used as a validation set to measure the classifier's performance (step 1). The training subset were then down-sampled to 500,000 counts per cell (weightedDown_total=5e5), then scaled up such that the total expression per cell was 100000 (transprop_xFact=1e5) and log transformed (step 2). Using log-transformed down-sampled counts, the top 25 differentially over-expressed genes, top 25 differentially under-expressed genes and 25 least differentially expressed genes were found as baseline genes for generating gene-pairs per cancer type (nTopgenes=25) (step 3). A quicker version of pair-transform different from Tan, et al (Tan et al. (2018)) “SingleCellNet: a computational tool to classify single cell RNA-Seq data across platforms and across species,” BioRxiv) (quickPairs=TRUE) was performed by generating gene-pairs among the 75 genes found in step 3 for each cancer type (step 4). The normalized training data were binarized through pair-transformation inspired by the top-pair classifier (Geman et al. (2004) “Classifying gene expression profiles from pairwise mRNA comparisons,” Statistical Applications in Genetics and Molecular Biology 3, p. Article19.). The top 70 most discriminatory gene-pairs for each cancer type were then selected (step 5) (Table 1). Additionally, 70 random gene-pair profiles were generated through random permutations of existing training data (nrand=70) annotated as “rand” or “Unknown” category in which is designed to capture cases where samples in query do not have representation in the cancer categories in the classifier (step 6). Using selected top gene-pairs as features, a CCN random forest classifier of 1000 trees (nTrees=1000) was constructed (step 7). Additionally, stratified sampling in the construction of random forest classifier was used with a strata size of 60 (stratify=TRUE, samplesize=60) to resolve the issue of imbalance profiles quantity across different cancer types.

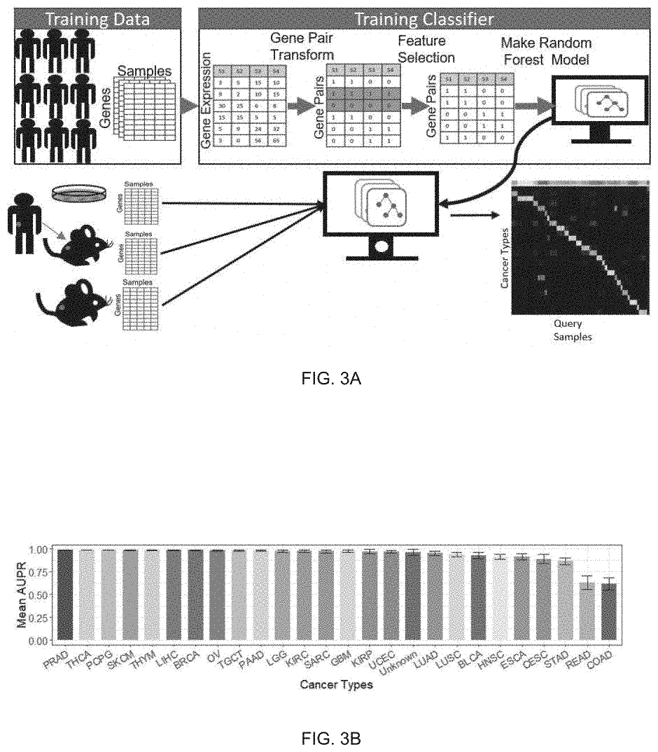

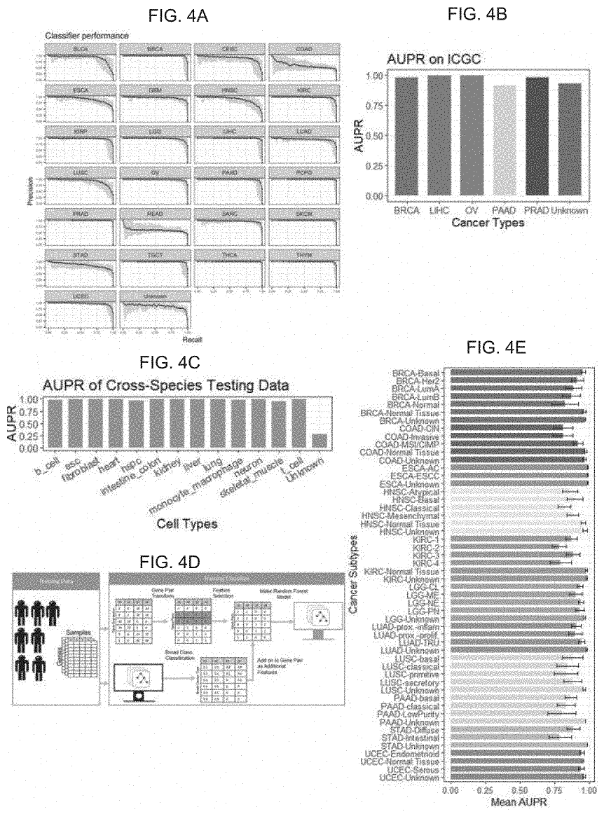

After the CCN classifier was built, 35 held-out samples from each of the cancer categories from the held-out data were randomly sampled and generated 40 “Unknown” profiles for validation (step 8). The held-out data was gene-pair transformed for assessment based on the top gene-pairs selected (step 9). The performance of the classifier was assessed by using precision-recall curve and area under the precision-recall curve (AUPR) (step 10). The process of randomly sampling a training set from all patient tumor data, train classifier and validate using validation set (step 1-10) was repeated 50 times to have a robust assessment of the classifier represented in FIG. 3B and FIG. 4A. After the parameters were tuned based on the performance of classifier on held-out data, a final version CCN classifier was trained using all the TCGA patient tumor data and 2000 trees (nTrees=2000) with all the other parameters staying the same to improve overall robustness and classification power. The specific parameters for the final CCN classifier and can gene-pairs be found in Table 1. The parameters used to train CCN are provided in Table 13.

Classifying Query Data into Broad Class

The cancer cell lines expression profiles and sample table were downloaded from a portal at the Broad Institute. PDX expression profiles and a sample table were obtained from Gao et al (Gao et al. (2015) “High-throughput screening using patient-derived tumor xenografts to predict clinical trial drug response,” Nature Medicine 21(11):1318-1325). GEMM expression profiles were obtained from 10 different studies on GEO database (Adeegbe et al. (2018) “BET Bromodomain Inhibition Cooperates with PD-1 Blockade to Facilitate Antitumor Response in Kras-Mutant Non-Small Cell Lung Cancer,” Cancer immunology research 6(10):1234-1245; Blaisdell et al. (2015), “Neutrophils oppose uterine epithelial carcinogenesis via debridement of hypoxic tumor cells,” Cancer Cell 28(6):785-799; Fitamant et al. (2015) “YAP inhibition restores hepatocyte differentiation in advanced HCC, leading to tumor regression,” Cell reports 10(10):1692-1707; Jia et al. (2018) “Crebbp loss drives small cell lung cancer and increases sensitivity to HDAC inhibition,” Cancer discovery 8(11):1422-1437; Kress et al. (2016) “Identification of MYC-Dependent Transcriptional Programs in Oncogene-Addicted Liver Tumors,” Cancer Research 76(12):3463-3472; Li et al. (2018) “GKAP acts as a genetic modulator of NMDAR signaling to govern invasive tumor growth,” Cancer Cell 33(4):736-751.e5; Mollaoglu et al. (2018) “The Lineage-Defining Transcription Factors SOX2 and NKX2-1 Determine Lung Cancer Cell Fate and Shape the Tumor Immune Microenvironment,” Immunity 49(4):764-779.e9; Pan et al. (2017) “Whole tumor RNA-sequencing and deconvolution reveal a clinically-prognostic PTEN/PI3K-regulated glioma transcriptional signature,” Oncotarget 8(32):52474-52487; Lissanu Deribe et al. (2018) “Mutations in the SWI/SNF complex induce a targetable dependence on oxidative phosphorylation in lung cancer,” Nature Medicine 24(7):1047-1057). To use CCN classifier on GEMM data, the mouse genes were converted from GEMM expression profiles into human orthologs. Once a final classifier was trained with all the patient tumor samples, the query samples were gene-pair transformed with gene-pairs selected from the training step and the query samples were classified using CCN. The results were analyzed using R and the classification results were visualized through heatmaps and attribution plots processed using R package ggplot2 (Wickham (2016) ggplot2—Elegant Graphics for Data Analysis. New York, N.Y.: Springer-Verlag New York).

Cross-Species Assessment

Among the innovative aspects of the CCN tool is the ability for cross species analysis. To assess the performance of cross-species classification, 1003 labelled human tissue/cell type and 1993 labelled mouse tissue/cell type RNA-seq expression profiles were downloaded from Github. The mouse genes were converted into human orthologous genes. Then the intersecting genes were found between mouse tissue/cell expression profiles and human tissue/cell expression profiles. Using the intersecting genes, a CCN classifier was trained with all the human tissue/cell expression profiles. The parameters can be found in Table 3. After the classifier was trained, 75 samples were randomly sampled from each tissue category in mouse tissue/cell data and the classifier was applied on those samples to assess performance. The AUPR is depicted in FIG. 4C.

Cross-Technology Assessment

To assess the performance of CCN in applications to microarray, 6219 patient tumor microarray profiles were gathered across 12 different cancer types from the GEO database from more than 100 different projects. The interesting genes between the microarray profiles and TCGA patient RNA-seq profiles were located. Using those genes as features, a CCN classifier was created with all the TCGA patient profiles using hyper-parameters listed in Table 4. The parameters used to train CCN are provided in Table 13. After the microarray specific classifier was trained, 60 microarray patient samples were randomly sampled from each cancer category, and the CCN classifier was applied on them as an assessment of the cross-technology performance. The same CCN classifier was used to classify microarray CCL samples.

Training Sub-Type CancerCellNet

Eleven cancer types (BRCA, COAD, ESCA, HNSC, KIRC, LGG, PAAD, UCEC, STAD, LUAD, LUSC) were found which have meaningful subtypes based on either histology or expression and sufficient samples in every subtype to train a sub-type classifier with high AUPR. Normal tissue samples were also included from BRCA, COAD, HNSC, KIRC, UCEC to create a normal tissue category in the construction of their sub-type classifier. To train a sub-type classifier, a sample table was manually curated annotating each as either a cancer sub-type or “Unknown” representing other cancer types. Similar to training for broad class classifier, ⅔ of all samples in each sub-type (and “Unknown” category) were randomly sampled as training data. Expression down sampling, gene selections, gene-pair transform and selection (step 2-5 from broad training) were performed using just the samples labelled as a cancer sub-type (excluding samples labelled as “Unknown”) to find discriminating gene pairs that can differentiate sub-type in the broad cancer. Different from the broad class CCN training, the quick version of pair-transform was not used for creating gene-pairs for feature selection. In addition to having gene-pairs as features, the final broad class classifier was applied to all the training samples and the classification scores were added as features to mainly discriminate between the broad cancer type of interest and other cancer types. For some sub-type classifiers, the weight of the broad classification scores were increased as features to fine tune the sub-type classifiers. Some random permutation samples were also generated to add to the “Unknown” training data along with expression profiles of other cancer types. The specific parameters used to train individual sub-type classifiers can be found in Table 5. The parameters used to train CCN are provided in Table 13.

An equal amount across all sub-types and Unknown category in the held-out data was then sampled for assessing the sub-type classifiers through AUPR. The process was repeated 20 times for robust assessment of the sub-type classifiers. The results are shown in FIG. 4E. For the final sub-type classifiers of the 11 broad categories, all of the TCGA data was used.

Classifying Query Data into Sub-Type

The 11 sub-type classifiers were applied on query samples when available. Heatmap visualizations were done using ComplexHeatmap package (Gu et al. (2016) “Complex heatmaps reveal patterns and correlations in multidimensional genomic data,” Bioinformatics 32(18):2847-2849) and other analysis were done in R.

Results

CancerCellNet Classifies Samples Accurately Across Species and Technologies

A computational tool was previously developed using the Random Forest classification method to measure the similarity of engineered cell populations with their in vivo counterparts based on transcriptional profiles (Cahan et al. (2014) “CellNet: network biology applied to stem cell engineering,”. Cell, 158(4):903-915.; Radley et al. (2017) “Assessment of engineered cells using CellNet and RNA-seq,” Nature Protocols 12(5):1089-1102). This approach was recently elaborated to allow for classification of single cell RNA-Seq data in a manner that allows for cross-platform and cross-species analysis (Tan et al. (2018) “SingleCellNet: a computational tool to classify single cell RNA-Seq data across platforms and across species,” BioRxiv.). In the present example, an approach was used to quantitatively compare cancer models to naturally occurring patient tumors (FIG. 3A). In brief, The Cancer Genome Atlas (TCGA) expression data was used from 25 solid tumor types to train a top-pair multi-class Random forest classifier. The approach also included an ‘Unknown’ category trained on a random shuffling and sampling of profiles from the remaining 24 tumor types in the training data to identify query samples that are not reflective of any of the training data.

The performance of this approach was assessed by computing the area under the precision recall curves derived by k-fold cross validation (n=50) (FIG. 3B and FIG. 4A). In the k-fold cross validation, the mean AUPR exceeded 0.95 in most of the tumor types and was below 0.7 only for the READ and COAD categories. This is not surprising as READ and COAD are considered to be the same disease. In addition to achieving high mean AUPRs on held-out TCGA data, it was found that CCN also achieved high AUPR (above 0.9) when it was applied to independent testing data from ICGC consisting RNA-Seq data from 886 tumors across 5 tumor types (FIG. 4B) (Zhang et al. (2011) “International Cancer Genome Consortium Data Portal—a one-stop shop for cancer genomics data,” Database: the Journal of Biological Databases and Curation, p. bar026).

One of the aims of the study was to compare distinct cancer models, including GEMMs, the exemplary method was able to classify samples from mouse and human samples equivalently. The Top-Pair transform, previously described (Tan et al. (2018) “SingleCellNet: a computational tool to classify single cell RNA-Seq data across platforms and across species,” BioRxiv), was used to achieve this and the feasibility of this approach was tested by assessing the performance of a normal (i.e., non-tumor) human tissue classifier as applied to mouse tissues. Consistent with prior applications, it was found that the cross-species classifier performed well, achieving mean AUPR of 0.93 when applied to mouse data (FIG. 4C).

To evaluate cancer models at a finer resolution, an approach was developed to perform tumor sub-type classifications (FIG. 4D). Eleven different cancer sub-type classifiers were constructed based on the availability of expression or histological subtype information (Cancer Genome Atlas Network (2012), “Comprehensive molecular portraits of human breast tumours,” Nature 490(7418):61-70; Parker et al. (2009), “Supervised risk predictor of breast cancer based on intrinsic subtypes,” Journal of Clinical Oncology 27(8): 1160-1167; Cancer Genome Atlas Network (2012), “Comprehensive molecular characterization of human colon and rectal cancer,” Nature 487(7407):330-337; Cancer Genome Atlas Research Network (2017), “Integrated genomic characterization of pancreatic ductal adenocarcinoma,” Cancer Cell 32(2):185-203.e13; Cancer Genome Atlas Network (2015), “Comprehensive genomic characterization of head and neck squamous cell carcinomas,” Nature 517(7536):576-582; Cancer Genome Atlas Research Network (2013), “Comprehensive molecular characterization of clear cell renal cell carcinoma,” Nature 499(7456):43-49; Verhaak et al. (2010), “Integrated genomic analysis identifies clinically relevant subtypes of glioblastoma characterized by abnormalities in PDGFRA, IDH1, EGFR, and NF1,” Cancer Cell 17(1):98-110; Cancer Genome Atlas Research Network (2014), “Comprehensive molecular profiling of lung adenocarcinoma,” Nature 511(7511): 543-550; Wilkerson et al. (2010), “Lung squamous cell carcinoma mRNA expression subtypes are reproducible, clinically important, and correspond to normal cell types,” Clinical Cancer Research 16(19):4864-4875; Cancer Genome Atlas Research Network, Analysis Working Group: Asan University, BC Cancer Agency, et al. (2017), “Integrated genomic characterization of oesophageal carcinoma,” Nature 541(7636):169-175; Hu et al. 2012; Cancer Genome Atlas Research Network, Kandoth et al. (2013) “Integrated genomic characterization of endometrial carcinoma,” Nature 497(7447):67-73). Non-cancerous, normal tissues were also included when available for several sub-type classifiers (BRCA, COAD, HNSC, KIRC and UCEC). The 11 sub-type classifiers all achieved high overall AUPRs ranging from 0.78 to 0.98 (FIG. 4E).

Fidelity of Cancer Cell Lines

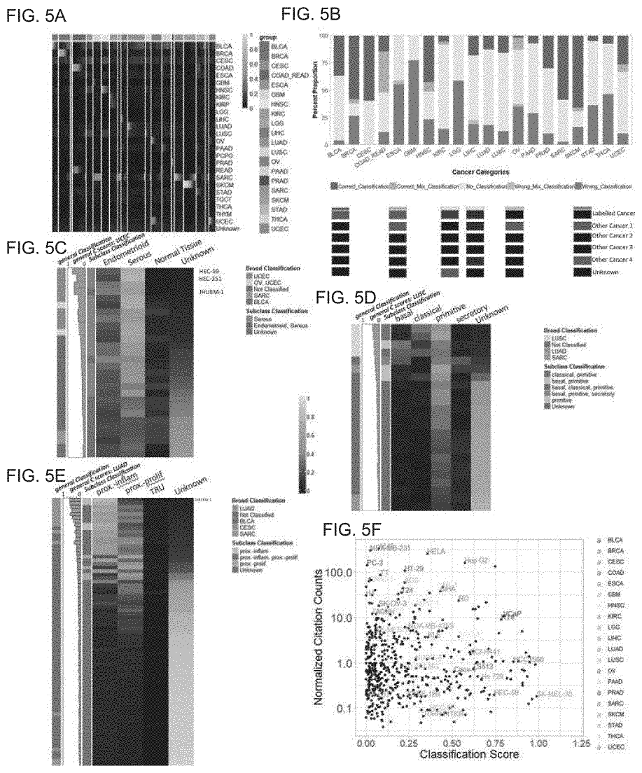

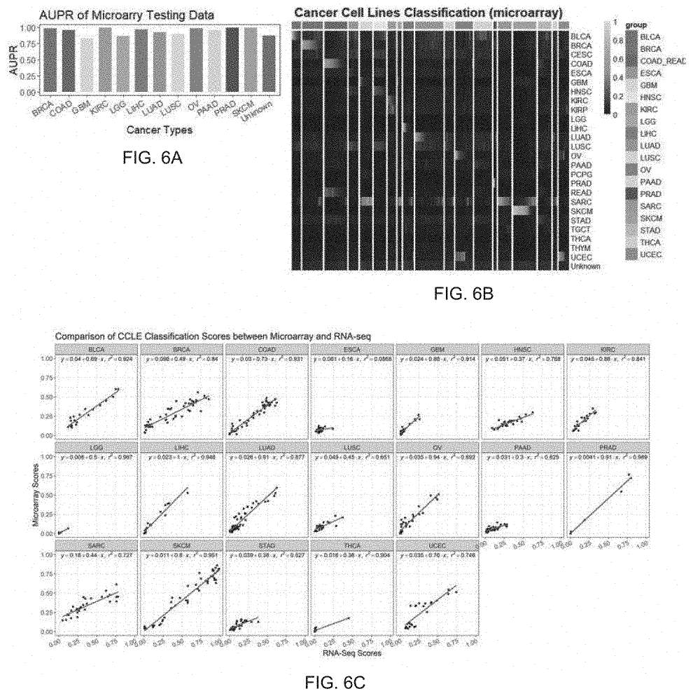

Having validated the performance of CCN, it was then used to determine the fidelity of CCLs. RNA-seq expression data of 657 different cell lines was mined across 20 cancer types from Cancer Cell Line Encyclopedia (CCLE) and CCN was applied to them, finding a wide classification range for cell lines of each tumor type (FIG. 5A). To verify the classification results, CCN was applied to CCLE expression profiles generated through microarray expression profiling. To ensure that CCN would function on microarray data, CNN was applied to 720 expression profiles of 12 tumor types from GEO. The cross-platform CCN classifier performed well, based on comparison to study-provided annotation, achieving a mean AUPRs of 0.94 (FIG. 6A). Next, this was applied cross-platform classifiers to microarray expression profiles of CCLE (FIG. 6B). From the classification results of 571 cell lines that have both RNA-seq and microarray expression profiles, a strong positive association was found between the classification scores from RNA-seq and those from microarray (FIG. 6C). This comparison supports the notion that the classification scores for each cell line are not artifacts of profiling methodology. Moreover, this comparison shows that the scores are consistent between the times that the cell lines were first assayed by microarray expression profiling in 2012 and by RNA-Seq in 2019, further validating the robustness of the CCN results.

Next, the CCN scores of CCLE cell lines was categorized based on the proportion of lines associated with each tumor type that were correctly classified. A decision threshold of 0.266 was set, which was selected as it represents the 5th percentile of all TCGA held-out classification scores to ensure at least 95% true positive rate for the held-out data. Each cell line was placed into one of five categories based on its CCN profile: correctly classified, mix-correctly classified, not classified, mix incorrectly classified and incorrectly classified (FIG. 5B). Cell lines originally annotated as BRCA, CESC SKCM and SARC had a high proportion of lines correctly classified. The COAD_READ cell lines had a high proportion of cell lines with mixed classification, reflecting the similarities of the tumor samples in the COAD and READ training data. Seventeen out of twenty tumor types had greater than 25% of lines that received no classification. In particular, no ESCA, GBM and LGG cell lines were classified as such, suggesting that these tumor types need more faithful cell line models (FIGS. 5 A and B).

One way to explain low classification scores is that some cell lines are derived from and represent sub-types of tumors that are not well-represented in TCGA. To explore this hypothesis, tumor sub-type classification was first performed on the CCLE lines from 11 tumor types for which sub-type classifiers had been trained. It was reasoned that if a cell was a good model for a rarer sub-type, then it would receive a poor general classification but a high classification for the sub-type that it models well. Therefore, the number of lines that fit this pattern was counted. It was found that of the 198 lines with no general classification, 52 (26%) were classified as a specific sub-type, suggesting that derivation from rare sub types is not the major contributor to poor overall CCL classification.

Another potential contributor to low scoring cell lines could be the intra-tumor impurity in the training data. If impurity were such a confounder of CCN scoring, then a positive correlation between mean purity and mean CCN classification of CCLE per general tumor type would be expected. However low Pearson correlation of 0.076 between the mean purity and mean CCN classification scores of CCLE was found, suggesting that tumor purity is not a major contributor to the low scoring of CCLEs (FIG. 5D).

Next, the sub-type classification of CCLs from three general tumor types was explored in more depth, focusing first on Uterine Corpus Endometrial Carcinoma (UCEC). The histological based sub-types of UCEC, endometrioid and serous histological type, differ in prevalence, molecular properties, prognosis, and treatment (Black et al. (2014), “Targeted therapy in uterine serous carcinoma: an aggressive variant of endometrial cancer,” Women's health (London, England) 10(1):45-57; Yang et al. (2011), “Progesterone: the ultimate endometrial tumor suppresso,” Trends in Endocrinology and Metabolism 22(4):145-152). CCN classified the majority of the UCEC cell lines as serous. All of the other lines were classified as ‘unknown’ except for JHUEM-1 and HEC-265, which received a mixed serous and endometrioid, meaning that the classification of each sub-type exceeded the 5th percentile of TCGA held-out classification scores (FIG. 5C). The preponderance of serous versus endometroid may be due to properties of serous cancer cells that aid propagation in vitro, such as upregulation in cell adhesion (Huszar et al. (2010), “Up-regulation of L1CAM is linked to loss of hormone receptors and E-cadherin in aggressive subtypes of endometrial carcinomas,” The Journal of Pathology 220(5):551-561) helps the derivation of CCLs. Some of the sub-type classification results are consistent with prior observations. For example, HEC-1A, HEC-1B, and KLE were previously characterized as endometrial (Kozak et al. (2018) “A guide for endometrial cancer cell lines functional assays using the measurements of electronic impedance,” Cytotechnology 70(1):339-350). On the other hand, the sub-type classification results contradict prior observations in at least one case. For example, Ishikawa ER− has been used as a model of endometroid cancer (Korch et al. (2012), “DNA profiling analysis of endometrial and ovarian cell lines reveals misidentification, redundancy and contamination,” Gynecologic Oncology 127(1):241-248; Kozak et al. (2018) “A guide for endometrial cancer cell lines functional assays using the measurements of electronic impedance,” Cytotechnology 70(1):339-350), CCN classified the Ishikawa 02 ER− cell line strongly as serous. This could be a result of ER negative being a characteristic of type 2 endometrial cancer (Black et al. (2014), “Targeted therapy in uterine serous carcinoma: an aggressive variant of endometrial cancer,” Women's health (London, England) 10(1): 45-57). Taken together, these results indicate a need for more endometroid-like CCLs.

Next, the sub-type classification of Lung Squamous Cell Carcinoma (LUSC) cell lines (FIG. 5D) was examined. It was found that of the 19 lines unclassified or misclassified in the general classifier, 16 (84%) were considered to be the unknown sub-type. These three lines had general classification scores modestly below the threshold; two had sub-type classification as primitive, and one as a mix of basal, primitive and secretory. Among all of the cell LUAD lines that were classified, all the cell lines have underlying primitive subtype classification. This is consistent either with the ease of deriving lines from tumors with a primitive character, or with a process by which cell line derivation promotes similarity to more the primitive sub-type, which is marked by increased cellular proliferation (Wilkerson et al. (2010), “Lung squamous cell carcinoma mRNA expression subtypes are reproducible, clinically important, and correspond to normal cell types,” Clinical Cancer Research 16(19):4864-4875). The results are consistent with prior reports that have investigated the resemblance of some lines to LUAD sub-types. For example, HCC-95, classified as classical and primitive subtype, has previously been characterized as classical (Wu et al. (2013), “Gene-expression data integration to squamous cell lung cancer subtypes reveals drug sensitivity,” British Journal of Cancer 109(6):1599-1608; Wilkerson et al. (2010), “Lung squamous cell carcinoma mRNA expression subtypes are reproducible, clinically important, and correspond to normal cell types,” Clinical Cancer Research 16(19):4864-4875). Further, LUDLU-1, classified as a mix of primitive, basal and classical, was previously characterized as resembling both basal and classical (Wu et al. (2013), “Gene-expression data integration to squamous cell lung cancer subtypes reveals drug sensitivity,” British Journal of Cancer 109(6):1599-1608). Lung Adenocarcinoma (LUAD) cell lines had classification results similar to LUSC: most lines did not classify as LUAD in the general classifier (53 of 76), and most of the remaining lines exhibited mixed sub-type classification (FIG. 5E). RERF-LC-Ad1 had the highest general classification score and the highest proximal inflammation sub-type classification score. Taken together, these sub-type classification results have revealed an absence of cell lines models for basal, classical, and secretory LUSC, and for the TRU LUAD sub-type.

Finally, it was sought to measure the extent to which cell line transcriptional fidelity related to model use. The number of papers in which a model was mentioned was used, normalized by the number of years since the cell line was derived, as a rough approximation of model usage. To explore this metric, the normalized citation count was plotted versus general classification score, labeling the highest cited and highest classified cell lines from each general tumor type (FIG. 5F). For most of the general tumor types, the highest cited cell line is not the highest classified cell line except for Hep G2 and ML-1, representing LIHC and THCA, respectively. On the other hand, the general scores of the highest cited cell lines representing BRCA, LUAD, OV, PRAD and SKCM fall below the classification threshold of 0.266. Notably, each of these tumor types have lines with scores exceeding 0.5, suggesting that these lines should be considered as more faithful transcriptional models when selecting lines for a study.

Evaluation of Patient Derived Xenografts

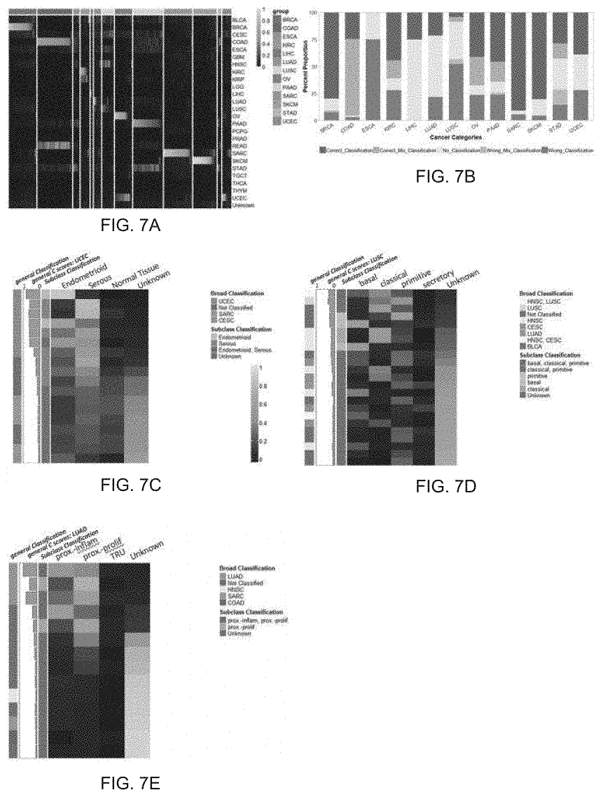

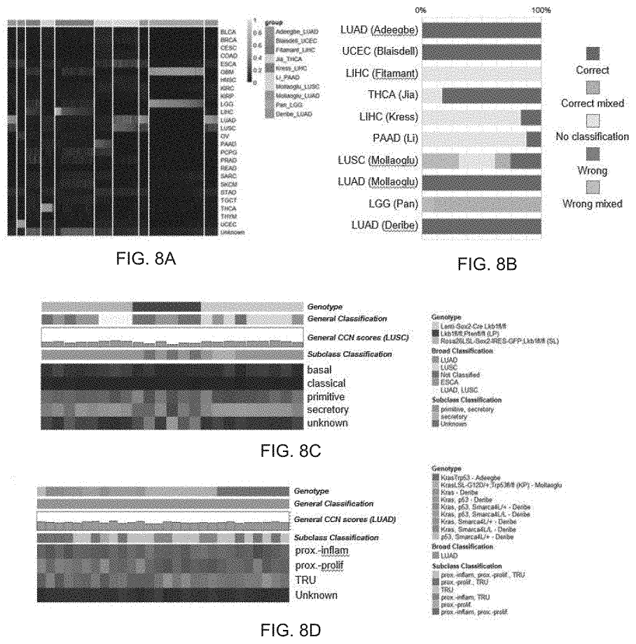

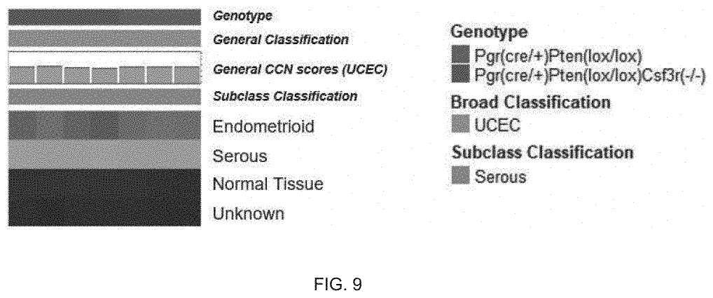

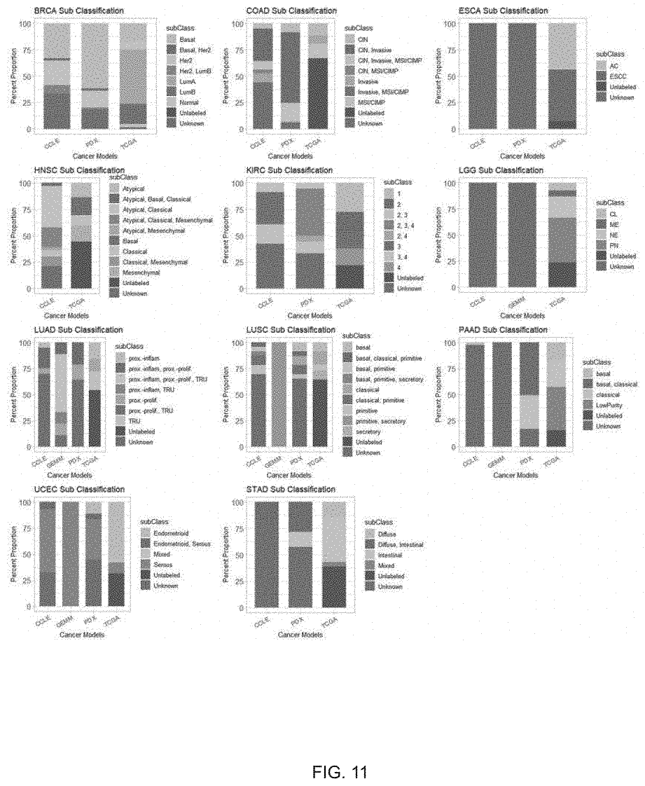

Next, it was sought to evaluate a more recent class of cancer models: PDX. To do so, the RNA-Seq expression profiles of 415 PDX models from 13 different types of cancer types generated previously (Gao et al. (2015), “High-throughput screening using patient-derived tumor xenografts to predict clinical trial drug response,” Nature Medicine 21(11):1318-1325) were subjected to CCN. Similar to the results of CCLE, the PDXs exhibited a wide range of classification scores (FIG. 7A). By categorizing the CCN scores of PDX based on the proportion of samples associated with each tumor type that were correctly classified, it was found that SARC, SKCM and BRCA have higher proportion of correctly classified PDX than those of other cancer categories (FIG. 7B). In contrast to CCLE, it was found a higher proportion of correctly classified PDX in STAD and KIRC (FIG. 7B). However, similar to CCLE, no ESCA PDXs correctly classified. This held true when sub-type classification was performed on PDX samples: none of the PDX in ESCA were classified as any rare ESCA subtypes (FIG. 11). UCEC PDXs had both endometrioid subtypes, serous subtypes, and mixed subtypes, which provides broader representation than in CCLE (FIG. 8C). LUSC PDXs had a large proportion HNSC misclassified, yet strong as basal and classical subtype classification (FIG. 8D). This could be due to result from the similarity in expression profiles of basal and classical subtypes of HNSC and LUSC (Walter et al. (2013), “Molecular subtypes in head and neck cancer exhibit distinct patterns of chromosomal gain and loss of canonical cancer genes,” Plos One 8(2):e56823; Wickham (2016) ggplot2—Elegant Graphics for Data Analysis, New York, N.Y.: Springer-Verlag New York). No LUSC PDXs lack were classified as the secretory subtype (FIG. 8D). While 9 of the LUAD PDX samples were classified as the unknown sub-type class classification, the remaining 5 classify as proximal proliferative or mixed proximal proliferative and proximal inflammatory (FIG. 9). Finally, similar to the CCLE, there were no TRU subtypes in the PDX cohort (FIG. 9). Collectively, these results indicate that PDXs can have very high transcriptional fidelity to both general tumor types and sub-types.

Evaluation of GEMMs

Next, CCN was used to evaluate GEMMs of six general tumor types from ten studies for which expression data was publicly available (Adeegbe et al. (2018) “BET Bromodomain Inhibition Cooperates with PD-1 Blockade to Facilitate Antitumor Response in Kras-Mutant Non-Small Cell Lung Cancer,” Cancer immunology research 6(10):1234-1245; Blaisdell et al. (2015), “Neutrophils oppose uterine epithelial carcinogenesis via debridement of hypoxic tumor cells,” Cancer Cell 28(6):785-799; Fitamant et al. (2015) “YAP inhibition restores hepatocyte differentiation in advanced HCC, leading to tumor regression,” Cell reports 10(10):1692-1707; Jia et al. (2018) “Crebbp loss drives small cell lung cancer and increases sensitivity to HDAC inhibition,” Cancer discovery 8(11):1422-1437; Kress et al. (2016) “Identification of MYC-Dependent Transcriptional Programs in Oncogene-Addicted Liver Tumors,” Cancer Research 76(12):3463-3472; Li et al. (2018) “GKAP acts as a genetic modulator of NMDAR signaling to govern invasive tumor growth,” Cancer Cell 33(4):736-751.e5; Mollaoglu et al. (2018) “The Lineage-Defining Transcription Factors SOX2 and NKX2-1 Determine Lung Cancer Cell Fate and Shape the Tumor Immune Microenvironment,” Immunity 49(4):764-779.e9; Pan et al. (2017) “Whole tumor RNA-sequencing and deconvolution reveal a clinically-prognostic PTEN/PI3K-regulated glioma transcriptional signature,” Oncotarget 8(32):52474-52487; Lissanu Deribe et al. (2018) “Mutations in the SWI/SNF complex induce a targetable dependence on oxidative phosphorylation in lung cancer,” Nature Medicine 24(7):1047-1057). As was true for CCLs and PDXs, GEMMs also had a wide range of CCN scores (FIG. 8A). The CCN scores were next categorized based on the proportion of samples associated with each tumor type that were correctly classified (FIG. 8B). In contrast to CCLs and PDXs, the GEMM dataset included multiple replicates per model, which allowed for the examination of intra-GEMM variability. Both at the level of CCN score and at the level of categorization, GEMMs were highly invariant. For example, replicates of LUAD GEMMs (driven by Kras mutation and loss of p53 (Adeegbe et al. (2018) “BET Bromodomain Inhibition Cooperates with PD-1 Blockade to Facilitate Antitumor Response in Kras-Mutant Non-Small Cell Lung Cancer,” Cancer immunology research 6(10):1234-1245), and Smarca4 loss (Lissanu Deribe et al. (2018) “Mutations in the SWI/SNF complex induce a targetable dependence on oxidative phosphorylation in lung cancer,” Nature Medicine 24(7):1047-1057), or overexpression of Sox2 and loss of Lkb1 (Mollaoglu et al. (2018) “The Lineage-Defining Transcription Factors SOX2 and NKX2-1 Determine Lung Cancer Cell Fate and Shape the Tumor Immune Microenvironment,” Immunity 49(4):764-779.e9) were all correctly classified (FIG. 8B). GEMMs sharing genotypes across studies, such as Pgr(cre/+)Pten(lox/lox)-driven UCEC (Blaisdell et al. (2015), “Neutrophils oppose uterine epithelial carcinogenesis via debridement of hypoxic tumor cells,” Cancer Cell 28(6):785-799; Daikoku et al. (2008) “Conditional loss of uterine Pten unfailingly and rapidly induces endometrial cancer in mice,” Cancer Research 68(14):5619-5627) received highly similar general and sub-type classification scores (FIG. 9). Even GEMMs with mixed classifications received consistent CCN scores. For example, LGG GEMMs, generated by Nf1 mutations expressed in different neural progenitors in combination with Pten deletion (Pan et al. (2017) “Whole tumor RNA-sequencing and deconvolution reveal a clinically-prognostic PTEN/PI3K-regulated glioma transcriptional signature,” Oncotarget 8(32):52474-52487), consistently received mixed classification as both LGG and GBM (FIG. 8A).

To explore the extent to which driver genotype impacts sub-type classification, two general tumor types were examined in which there were GEMMs with different tumor drivers: LUSC and LUAD. The LUSC GEMMs were generated using loss of Lkb1 and either overexpression of Sox2 (via two distinct mechanisms) or loss of Pten (Mollaoglu et al. (2018) “The Lineage-Defining Transcription Factors SOX2 and NKX2-1 Determine Lung Cancer Cell Fate and Shape the Tumor Immune Microenvironment,” Immunity 49(4):764-779.e9). It was found that most of the lenti-Sox2-Cre-infected;Lkb1^{fl/fl} samples were classified as LUSC, whereas the majority of the Rosa26LSL-Sox2-1RES-GFP;Lkb1^{fl/fl} samples were classified as either LUAD or a mixture of LUAD and LUSC (FIG. 8C). It is possible that the distinct transcriptional programs result from differing levels of exogenous Sox2 expression in these models, and that the samples with mixed classification results reflect an adenosquamous carcinoma phenotype. Most of the Lkb1^{fl/fl};Pten^{fl/fl} GEMMs were classified as ‘unknown’. Moreover, the sub-type classification indicated that this GEMM was either unknown or of mixed serous/primitive sub-type, in contrast to prior reports suggesting that it is most similar to a basal subtype (Xu et al. (2014) “Loss of Lkb1 and Pten leads to lung squamous cell carcinoma with elevated PD-L1 expression,” Cancer Cell 25(5):590-604). The results have shown that Lkb1fl/fl,Ptenfl/fl GEMMs are mostly classified as unknown and primitive, secretory subtypes which correlates with the general classification scores. The lenti-Sox2-Cre-infected;Lkb1^{fl/fl} samples were more strongly classified as the secretory sub-type, whereas the Rosa26LSL-Sox2-1RES-GFP;Lkb1^{fl/fl} samples were classified as a more balanced mix of serous and primitive sub-types. None of the three LUSC GEMMs were sub-typed as classical or basal. All of the LUAD GEMMs, which were generated using various combinations of activating Kras mutation, loss of Trp53, loss of Lkb1, and loss of Smarca4L (Lissanu Deribe et al. (2018) “Mutations in the SWI/SNF complex induce a targetable dependence on oxidative phosphorylation in lung cancer,” Nature Medicine 24(7):1047-1057; Adeegbe et al. (2018) “BET Bromodomain Inhibition Cooperates with PD-1 Blockade to Facilitate Antitumor Response in Kras-Mutant Non-Small Cell Lung Cancer,” Cancer immunology research 6(10):1234-1245); Mollaoglu et al. (2018) “The Lineage-Defining Transcription Factors SOX2 and NKX2-1 Determine Lung Cancer Cell Fate and Shape the Tumor Immune Microenvironment,” Immunity 49(4):764-779.e9), were correctly classified (FIG. 8D). There were no substantial differences in general, or sub-type classification across driver genotypes. Notably, the sub-types tended to be a mixture of proximal proliferation, proximal inflammation and TRU. Taken together, this analysis suggests that there is a degree of similarity, and perhaps plasticity between the primitive and secretory (but not basal or classical) sub-types of LUSC. On the other hand, while the LUAD GEMMs classify strongly as LUAD, all have a mixed sub-type classification—a result that does not vary by genotype.

Comparison of CCLs, PDXs, and GEMMs

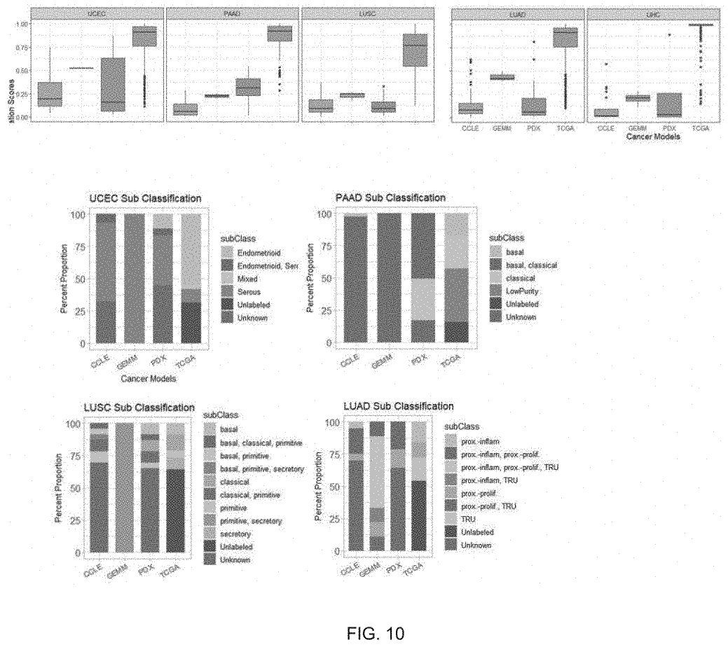

Finally, it was sought to estimate the comparative transcriptional fidelity of the three cancer models modalities, limiting the comparison to those five general tumor types for which there were at least two examples per modality: UCEC, PAAD, LUSC, LUAD, and LIHC. The general CCN scores of each model were compared on a per tumor type basis (FIG. 10). In the case of GEMMs, the mean classification score of all samples with shared genotypes was used. It was found that GEMMs had the highest median general classification scores in four out of the five tumor types. However, some PDXs achieved the highest classification scores. In UCEC, LUAD and LIHC, the maximum classification score of PDXs exceeded 0.75 and were thus comparable to the majority of scores on held out TCGA data, highlighting the potential for PDXs to mirror the transcriptional state of natural tumors (FIG. 10).

It was also sought to compare model modalities in terms of the diversity of sub-types that they represent. As a reference, the overall sub-type incidence was also included in this analysis, as approximated by incidence in TCGA. In models of UCEC, there is a notable difference in endometroid incidence, and the proportion of models classified as endometroid, with only PDX having any representatives (FIG. 10). The vast majority of CCLE and all of the GEMM models of PAAD have an unknown sub-type classification. However, the PDXs are sub-typed as either a mixture of basal and classical, or classical alone. No model of LUSC was sub-typed exclusively as secretory, and only PDXs were sub-typed exclusively as basal. No model of LUAD was sub-typed exclusively as TRU, but there were models that were sub-typed exclusively as proximal proliferative in both PDXs and GEMMs. Taken together, these results indicate that only a few CCLs are good transcriptional exemplars of natural tumor sub-types, that GEMMs are typically mixtures of sub-types, and the PDXs are the modality that can best reflect specific sub-types.

Discussion

A major goal in the field of cancer biology is to develop models that mimic naturally occurring tumors with enough fidelity to enable therapeutic discoveries. However, methods to measure the extent to which cancer models resemble or diverge from native tumors are lacking. This is especially problematic now because there are many existing models from which to choose, and it has become easier to generate new models. Accordingly, in certain aspects, this disclosure presents CancerCellNet (CCN), a computational tool that measures the similarity of cancer models to 25 naturally occurring tumor types and 46 sub-types. Because CCN is platform and species agnostic, it can be applied across many model modalities, including CCLs, PDXs, and GEMMs, and thus it represents a consistent platform to compare models across modalities. In this example, CCN was applied to 657 cancer cell lines, 415 patient derived xenografts, and 26 distinct genetically engineered mouse models. Several exemplary lessons emerged from these computational analyses that have implications for the field of cancer biology.

First, CancerCellNet indicates that GEMMs are transcriptionally the most faithful models of four out of five general tumor types for which data from all modalities was available. This is consistent with the fact that GEMMs are typically derived by recapitulating well defined driver mutations of natural tumors, and thus this observation corroborates the importance of genetics in the etiology of cancer. Moreover, in contrast to PDXs, GEMMs are typically generated in immune replete (complete) hosts. Therefore, the higher fidelity of GEMMs may also be a result of the influence of a native immune system on GEMM tumors. Second, PDXs and CCLs have lower scores that are comparable to each other. This is consistent with the observation that PDXs can undergo selective pressures in the host that distort the progression of genomic alterations away from what is observed in natural tumors (Ben-David et al. (2017) “Patient-derived xenografts undergo mouse-specific tumor evolution,” Nature Genetics 49(11):1567-1575). Furthermore, the observation that a few PDXs have very high classification scores, approaching a level that is indistinguishable from held out TCGA data, suggests that under certain conditions, PDX can almost perfectly mimic natural tumors transcriptionally. It is unclear what these conditions are; it may be that these few PDXs were profiled prior to the acquisition of non-typical genomic alterations. Third, it was found that none of the samples that we evaluated here are transcriptionally adequate models of ESCA, and therefore this tumor type requires further attention to derive new models. Fourth, it was found that in several tumor types, GEMMs tend to reflect mixtures of sub-types rather than conforming to single sub-types. The reasons for this are not clear but it is possible that in the cases that were examined, the histologically defined sub-types have a degree of plasticity that is exacerbated in the murine host environment.

CCN includes various embodiments or aspects. For example, CCN is based on transcriptomic data in some embodiments, but other molecular readouts of tumor state are also optionally utilized in lieu of, or in combination with, transcriptomic data, such as profiles of the proteome, epigenome, non-coding RNA-ome, and genome, among others, can also be mimicked in a model system. It is possible that some models reflect tumor behavior well, and because this behavior is not well predicted by transcriptome alone, these models have lower CCN scores. To both measure the extent that such situations exist, and to correct for them, other omic data is optionally incorporated into CCN so as to make more accurate and integrated model evaluation possible. Further, in the cross-species analysis, CCN generally implicitly assumes that homologs are functionally equivalent. The extent to which they are not functionally equivalent determines how confounded the CCN results will be. However, this possibility may be of limited consequence based on the high performance of the normal tissue cross-species classifier, and based on the fact that GEMMs have the highest median CCN scores. In addition, the TCGA training data is made up of RNA-Seq from bulk tumor samples, which necessarily includes non-tumor cells, whereas the CCLs are by definition cell lines of tumor origin. Therefore, CCLs theoretically could have artificially low CCN scores due to the presence of non-tumor cells in the training data. This potential problem appears to be limited as no correlation between tumor purity and CCN score was found in the CCLE samples. However, this potential problem may be related to the question of intra-tumor heterogeneity. Thus, in certain embodiments, CCN can be extended to interpret single cell RNA-Seq data. A sufficient amount of training single cell RNA-Seq data enables CCN to not only evaluate models on a per cell type basis, but also based on cellular composition.

TABLE 1
Gene Pairs For General Tumor Types

| BRCA | | GBM | | OV | | LUAD | | UCEC | |
|---|---|---|---|---|---|---|---|---|---|
| BRCA_1 | BRCA_2 | GBM_1 | GBM_2 | OV_1 | OV_2 | LUAD_1 | LUAD_2 | UCEC_1 | UCEC_2 |
| LMX1B | MIB2 | PSRC1 | FLNB | WT1 | TAF15 | NAPSA | PPP2R1A | DLX5 | PRNP |
| LMX1B | ANKS6 | KLHDC8A | FLNB | WT1 | SUN2 | SFTA2 | ITPK1 | DLX6 | NR3C1 |
| LMX1B | ID1 | C21orf62 | NET1 | WT1 | DST | SFTA2 | OAF | DLX5 | SBDS |
| TRPS1 | ODC1 | NR2E1 | NET1 | KCNK15 | ORMDL3 | SFTA2 | PLCD3 | DLX5 | RNF13 |
| PRLR | ETS2 | LCTL | FAM83H | KLHL14 | ORMDL3 | NAPSA | PTMS | MSX1 | SBDS |
| AARD | ANKS6 | GAP43 | NUCKS1 | ZNF503 | TAF15 | NAPSA | HNRNPC | DLX6 | TBC1D2B |
| TRPS1 | HADHA | PSRC1 | TRIM27 | KCNK15 | RETSAT | ROS1 | SLC16A1 | DLX6 | LYPLAL1 |
| TRPS1 | EIF3L | CNR1 | NET1 | KLHL14 | USP47 | SFTPD | CELSR2 | MSX1 | CALCOCO2 |
| PRLR | ODC1 | PSRC1 | HTATSF1 | KCNK15 | DNAJC3 | ROS1 | CELSR2 | MSX1 | TACC1 |
| IRX5 | ESRRA | RNASE2 | FAM83H | KLHL14 | DNAJC7 | SCGB3A2 | SLC16A1 | MAP2K6 | TAOK3 |
| AARD | PSAT1 | C21orf62 | DSTYK | ZNF503 | NAP1L4 | SFTPA1 | CELSR2 | STX18 | CALCOCO2 |
| EFHD1 | ITM2C | RFX4 | HTATSF1 | DOK5 | DST | ROS1 | PHGDH | STX18 | SERINC3 |
| IRX5 | MIB2 | RNASE2 | DSP | ATP6V1B1 | ORMDL3 | SFTPA1 | PHGDH | SOX17 | CREBL2 |
| IRX5 | ID1 | NR2E1 | NT5DC1 | DOK5 | SPAG9 | SFTPC | HR | STX18 | TM9SF4 |
| AARD | FZD5 | PLA2G5 | MYO1D | ATP6V1B1 | NAP1L4 | BPIFA1 | HR | SOX17 | PRNP |
| PRLR | ETFB | NR2E1 | LSR | ZNF503 | NBR1 | SFTPA1 | SOX9 | CCDC157 | LYPLAL1 |
| GATA3 | GSTP1 | LCTL | BAIAP2L1 | DOK5 | ABR | SFTPD | ECSIT | TEKT2 | LYPLAL1 |
| GATA3 | ITM2C | C21orf62 | MYO1D | ATP6V1B1 | TAF15 | SFTPD | TIMM44 | SOX17 | TBC1D2B |
| TBC1D9 | HADHA | LCTL | DSP | PNOC | PPP3CC | COL6A5 | HR | MAP2K6 | SBDS |
| PIP | PSAT1 | PLA2G5 | LSR | NPR1 | NBR1 | SCGB3A2 | PHGDH | FGF18 | PLSCR4 |
| GATA3 | ETS2 | PLA2G5 | KIAA1217 | LYPD1 | DST | SFTPC | SLC16A1 | FGF18 | NR3C1 |
| EFHD1 | CKB | HEPACAM | HTATSF1 | LYPD1 | NAP1L4 | SFTPC | SYNGR1 | MAP2K6 | RNF13 |
| PLEKHF2 | ODC1 | RNASE2 | LSR | NPR1 | SUN2 | SCGB3A2 | LARP6 | ARMC3 | PLSCR4 |
| CILP | ITM2C | POU3F2 | BRD3 | PNOC | ELL2 | LGSN | PLEKHH1 | ARMC3 | NR3C1 |
| SLC16A6 | ANKS6 | GAP43 | CNDP2 | PNOC | NIPA1 | TREM1 | OAF | FGF18 | NEDD4 |
| NAT1 | UBE2E3 | KLHDC8A | JUP | NPR1 | SPAG9 | TREM1 | PLCD3 | HOXB6 | CALCOCO2 |
| ESR1 | GSTP1 | CNR1 | FLNB | LYPD1 | SPAG9 | LGSN | PPT2 | TEKT2 | PLSCR4 |
| FSIP1 | STARD4 | RNASE3 | HOOK1 | DOK7 | LRP11 | CCNJL | ECSIT | EMX2 | PRNP |
| PIP | FZD5 | MT3 | NUCKS1 | DOK7 | TMEM181 | CCNJL | TIMM44 | RNF183 | ADCY9 |
| PIP | MID1 | POU3F2 | DSTYK | RSPO1 | LRP11 | SFTPB | PPP2R1A | ELP3 | SERINC3 |
| SERTAD4 | RNF145 | KLHDC8A | MYO1C | RSPO1 | PPP3CC | LPCAT1 | PPP2R1A | RNF183 | TAOK3 |
| NAT1 | RNF145 | RNASE3 | ARHGEF5 | RSPO1 | STK39 | SFTPB | PTMS | EMX2 | MAF |
| NAT1 | PPARA | CNR1 | DSTYK | DOK7 | TOM1 | TBX4 | SYNGR1 | EMX2 | CREBL2 |
| FSIP1 | PRKCA | DBX2 | DSP | MEIS1 | NBR1 | SFTPB | HNRNPC | C2orf88 | TACC1 |
| FSIP1 | PSAT1 | RFX4 | BRD3 | CTU1 | STK39 | NKX2-1 | WIZ | RNF183 | ELL2 |
| CILP | RNF145 | RNASE3 | BAIAP2L1 | MEIS1 | SUN2 | LGSN | OAF | DACT2 | FKBP5 |
| SLC16A6 | PPARA | DBX2 | HOOK1 | SOX17 | ABR | NKX2-1 | TIMM44 | ASRGL1 | SERINC3 |
| SLC16A6 | SLC9A6 | S100B | NUCKS1 | SOX17 | DNAJC7 | NKX2-1 | ECSIT | C2orf88 | TAOK3 |
| TBC1D9 | EIF3L | GAP43 | WFS1 | CTU1 | LRP11 | TBX4 | LARP6 | HOXB6 | TACC1 |
| CILP | ETS2 | GFAP | JUP | SOX17 | GGNBP2 | MUC21 | PLCD3 | HOXB6 | SETD7 |
| EFHD1 | BIN1 | DBX2 | NT5DC1 | HTR3A | STK39 | BMP5 | PPT2 | TEKT2 | ADCY9 |
| ST8SIA6 | PRKCA | GFAP | MYO1C | HTR3A | ELL2 | LPCAT1 | HNRNPC | DACT2 | CREBL2 |
| LRRC15 | PFKP | GFAP | STAT6 | KLK7 | ABR | CCNJL | ERF | ARMC3 | NEDD4 |
| SERTAD4 | UBE2E3 | PMP2 | PERP | HTR3A | NIPA1 | BMP5 | LDLRAD3 | C2orf88 | RNF13 |
| ST8SIA6 | FZD5 | PMP2 | MYO1D | MAMSTR | PPP3CC | BMP5 | PLEKHH1 | ASRGL1 | USP22 |
| SERTAD4 | PITPNM1 | POU3F2 | GTF3C4 | IMPG2 | GALK2 | BPIFA1 | LARP6 | DACT2 | SETD7 |
| ST8SIA6 | STARD4 | PMP2 | LTBR | UPK3B | ELL2 | XKRX | PLEKHH1 | CCDC157 | ADCY9 |
| LRRC15 | PITPNM1 | MT3 | STAT6 | MEIS1 | DNAJC3 | TREM1 | ERF | CCDC157 | NEDD4 |
| LRRC15 | GSTP1 | MT3 | MYO1C | LRRTM1 | NIPA1 | BPIFA1 | SYNGR1 | HOXB8 | SETD7 |
| SCUBE2 | CKB | RFX4 | BAIAP2L1 | CTU1 | GALK2 | MUC21 | KAZN | ASRGL1 | TM9SF4 |
| TFAP2B | BMP2 | MLC1 | JUP | UPK3B | TOM1 | TBX4 | PPT2 | FOXJ1 | MFSD1 |
| GFRA1 | BIN1 | HEPACAM | FAM83H | KLK7 | AHR | LPCAT1 | PTMS | CCDC114 | RAB8B |
| TFAP2B | BIN1 | MLC1 | SPINT2 | KLK7 | CALCOCO2 | MUC21 | SOX9 | CCDC114 | FKBP5 |
| STC2 | PFKP | HEPACAM | KRT8 | MAMSTR | GALK2 | MBIP | GTF2F1 | HOXB8 | MFSD1 |
| GFRA1 | HADHA | MLC1 | LTBR | LRRTM1 | USP47 | SCGB3A1 | KAZN | CCDC114 | MAF |
| TFAP2B | CAPN5 | AQP4 | DDX5 | LRRTM1 | CAMK2D | MBIP | ITPK1 | FOXJ1 | FKBP5 |
| TBC1D9 | LAMC1 | SCRG1 | LTBR | UPK3B | DNAJC7 | PIP5KL1 | KAZN | ELP3 | TM9SF4 |
| PLEKHF2 | UBE2E3 | FOXG1 | KRT8 | FGF18 | HARS2 | MBIP | ERF | FOXJ1 | ELL2 |
| GFRA1 | CKB | AQP4 | SPINT2 | FGF18 | CAMK2D | PIP5KL1 | LDLRAD3 | HOXB8 | FBXL3 |
| PLEKHF2 | PFKP | SCRG1 | STAT6 | FGF18 | TMEM181 | SCGB3A1 | LDLRAD3 | C20orf85 | ELL2 |
| ESR1 | LAMC1 | FOXG1 | BRD3 | RPL17 | CALCOCO2 | COL6A5 | PSPN | C20orf85 | MAF |
| ESR1 | EIF3L | FOXG1 | KRT18 | CLDN16 | CAMK2D | XKRX | TLN2 | C20orf85 | TBC1D2B |
| SCUBE2 | ID1 | SCRG1 | SPINT2 | CLDN16 | RETSAT | SCGB3A1 | SOX9 | ELP3 | MFSD1 |
| STC2 | LAMC1 | ST8SIA5 | NT5DC1 | IMPG2 | SLC38A9 | C16orf89 | TLN2 | CCDC33 | CHRNE |
| SCUBE2 | ETFB | AQP4 | KRT18 | CTCFL | AHRR | CXCL17 | ITPK1 | CCDC33 | ARHGEF33 |
| AZGP1 | ETFB | BAALC | B4GALT1 | CLDN16 | USP47 | C16orf89 | GTF2F1 | CCDC33 | ZNF519 |
| AZGP1 | ESRRA | ST8SIA5 | KRT18 | RPL17 | DNAJC3 | C16orf89 | STK11 | TEKT4 | WDR44 |
| AZGP1 | CAPN5 | BAALC | CNDP2 | IMPG2 | CHIC1 | CXCL17 | GTF2F1 | TEKT4 | RAB8B |
| STC2 | PITPNM1 | KCNIP1 | KRT8 | CTCFL | TNFRSF4 | CXCL17 | WIZ | TEKT4 | TUBD1 |
| DCAF10 | SSBP1 | KCNIP1 | PERP | MAMSTR | HARS2 | COL6A5 | IL24 | WFDC2 | USP22 |

| KIRC | | HNSC | | LGG | | THCA | | LUSC | |
|---|---|---|---|---|---|---|---|---|---|
| KIRC_1 | KIRC_2 | HNSC_1 | HNSC_2 | LGG_1 | LGG_2 | THCA_1 | THCA_2 | LUSC_1 | LUSC_2 |
| TLR3 | SMARCD2 | ALOXE3 | ACP6 | KCNJ10 | ANXA2 | TG | RPN2 | SFTPA1 | SORBS2 |
| ENPP3 | FASN | SDR9C7 | SVIP | KCNJ10 | CLIC1 | TG | PRKCSH | EGFL6 | CXXC5 |
| TLR3 | RBM15B | HEPHL1 | ACP6 | KCNJ10 | MYL12B | TG | YWHAG | SFTPA1 | ME3 |
| SEMA5B | FASN | SDR9C7 | ACP6 | KCNJ9 | PDLIM1 | TPO | PYCR1 | ABCA13 | KIF13B |
| GAL3ST1 | GIPC1 | HEPHL1 | DDAH1 | CDH20 | OSTC | TPO | TMEM97 | SFTPA1 | FHIT |
| SEMA5B | SCAP | HEPHL1 | ADCY6 | GPR37L1 | TAGLN2 | TPO | METTL8 | ABCA13 | CXXC5 |
| TLR3 | FOXK2 | SDR9C7 | ICA1 | IL17D | OSTC | CRYGN | RACGAP1 | ABCA13 | MAGI1 |
| ENPP3 | SMARCD2 | ALOXE3 | SVIP | OLIG2 | CLIC1 | CRYGN | TMEM97 | RASSF9 | CXXC5 |
| GAL3ST1 | SCAP | KRTDAP | SVIP | OLIG1 | ANXA2 | CRYGN | NUSAP1 | ABCC5 | PEBP1 |
| ENPP3 | HMGA1 | KRTDAP | FN3K | KCNJ9 | TEAD3 | IYD | SCD | RASSF9 | ALDH7A1 |
| GAL3ST1 | RANGAP1 | KRTDAP | FARP1 | APC2 | TAGLN2 | DAPK2 | SCD | RASSF9 | CRIP2 |
| ESM1 | SEC13 | FAM25A | FN3K | PSD2 | OSTC | MUC15 | SCD | TP63 | CST3 |
| SEC14L6 | HMGB3 | ALOXE3 | ICA1 | CDH20 | PPCS | IYD | IRAKI | TP63 | PEBP1 |
| ESM1 | FASN | SLC10A6 | ICA1 | PSD2 | MYL12A | DAPK2 | IDH2 | ADH7 | KIF13B |
| SEMA5B | RANGAP1 | FAM25A | PPP1R9A | OLIG2 | ANXA2 | DAPK2 | MPZL1 | ADH7 | OASL |
| MTCP1 | ARHGAP39 | SBSN | FARP1 | OLIG1 | TAGLN2 | IYD | IDH2 | EGFL6 | CRIP2 |
| ESM1 | SCAP | IL36G | FN3K | CDH20 | PDLIM1 | MUC15 | IRAKI | ADH7 | ALDH7A1 |
| CLEC18B | ARHGAP39 | IL36G | HNMT | CACNG7 | CLIC1 | HHEX | IRAKI | ADAM23 | CRIP2 |
| SLC5A10 | BDH1 | CNFN | PKN1 | KCNJ9 | F11R | TCERG1L | SLC6A8 | GPR87 | CAMK2N1 |
| ENPEP | SEC13 | RNF222 | PPP1R9A | PSD2 | S100A11 | HHEX | IDH2 | TP63 | THRAP3 |
| CUBN | GIPC1 | PLA2G4E | HNMT | MMD2 | TEAD3 | INPP5J | PAICS | FBXO27 | ALDH7A1 |
| ENPEP | RANGAP1 | IL36RN | HNMT | APC2 | MYL12B | HHEX | PAICS | ADAM23 | MGRN1 |
| MTCP1 | BDH1 | IL36RN | DDAH1 | ZDHHC22 | PDLIM1 | MUC15 | PAICS | NTS | PLEKHA6 |
| ALPK2 | HMGA1 | SLC10A6 | ZNF253 | MMD2 | SERINC2 | SRL | SLC6A8 | ADAM23 | MXD4 |
| SEC14L6 | HMGA1 | IL36G | ZNF253 | TNR | MYL12B | SLC26A7 | SLC6A8 | GPR87 | PLEKHA6 |
| CLEC18B | BDH1 | SBSN | PKN1 | OLIG2 | S100A11 | INPP5J | MPZL1 | B3GNT5 | PNPLA2 |
| SLC5A10 | HMGB3 | CNFN | PATZ1 | RFX4 | S100A11 | LCN12 | DNPEP | ABCC5 | GDI2 |
| ALPK2 | GNL3 | IL36RN | ADCY6 | IL17D | PPCS | TCERG1L | UCK2 | FBXO27 | PNPLA2 |
| CUBN | MAVS | BNC1 | IVD | MMD2 | TES | TCERG1L | FAM189B | EGFL6 | MAGI1 |
| CD70 | HMGB3 | SBSN | IVD | ZDHHC22 | MYO1C | MGAT4C | ARHGEF9 | NTS | PNPLA2 |
| ALPK2 | FOXK2 | DSG1 | DDAH1 | GPR37L1 | MYL12A | WDR86 | FAM189B | GPR87 | KIF13B |
| CUBN | SMARCD2 | CNFN | FARP1 | RFX4 | MYL12A | INPP5J | KIAA0930 | ABCC5 | CST3 |
| COL23A1 | GIPC1 | PLA2G4E | ADCY6 | TNR | WBP11 | SLC26A7 | TMEM97 | ARTN | HDAC11 |
| SLC5A10 | RRP9 | FAM25A | ZNF253 | ATP6V1G2 | MYO1C | SLC26A7 | NUSAP1 | B3GNT5 | DDAH1 |
| ENPEP | COX7A2L | BNC1 | PKN1 | DSCAM | TEAD3 | LCN12 | TTLL12 | NTS | SORBS2 |
| TMEM72 | ARHGAP39 | DSG1 | TCTA | IL17D | MAP2K3 | ZCCHC12 | KIAA0930 | DSG3 | CAMK2N1 |
| CLEC18B | ZMYND19 | BNC1 | PATZ1 | ATP6V1G2 | TMEM214 | SRL | RACGAP1 | DSG3 | MGRN1 |
| SLC5A12 | ZNRF1 | PLA2G4E | P4HTM | ZDHHC22 | PPCS | WDR86 | TTLL12 | ARTN | MAGI1 |
| ZNF395 | EIF3E | KRT75 | CHKA | DSCAM | MAP2K3 | SRL | EIF4EBP1 | DSG3 | RNPEP |
| COL23A1 | SEC13 | DSG1 | CRELD1 | ATP6V1G2 | ZDHHC5 | C2orf40 | TMCO3 | DCUN1D1 | MGRN1 |
| ASPA | FOXK2 | SPRR2D | P4HTM | CMTM5 | LTBR | ZBED2 | TTLL12 | ARTN | SDSL |
| SLC5A12 | ZMYND19 | DSG3 | GNB1 | DSCAM | MYO1C | LCN12 | KIAA0930 | GCLC | CST3 |
| SLC5A12 | DOLPP1 | SLC10A6 | PPP1R9A | TNR | TMEM214 | C2orf40 | DNPEP | PTHLH | CAMK2N1 |
| SLC22A2 | DOLPP1 | FAM83C | P4HTM | CMTM5 | MAP2K3 | S100A5 | NUSAP1 | GCLC | PEBP1 |
| TMEM72 | PWWP2B | SPRR2D | PATZ1 | CACNG7 | TMEM214 | WDR86 | MRPL16 | PTHLH | RPS27L |
| ASPA | KIF22 | KRT75 | TMEM8B | RFX4 | VAMP8 | TMEM233 | EIF4EBP1 | KRT74 | RAPSN |
| SLC22A2 | ZMYND19 | NIPAL4 | SCCPDH | CACNG7 | PRKAG1 | NKX2-1 | YWHAG | FBXO27 | HDAC11 |
| SLC22A2 | RRP9 | SPRR2D | MGAT4A | CMTM5 | VAMP8 | C2orf40 | FAM189B | B3GNT5 | RPS27L |
| SEC14L6 | ZNRF1 | FGFBP1 | GORASP2 | OLIG1 | STAT6 | ZCCHC12 | YWHAG | PTHLH | PGPEP1 |
| COL23A1 | DYNLRB1 | KRT75 | PGPEP1 | CRB1 | LTBR | ZCCHC12 | ATAD1 | WDR53 | HDAC11 |
| CD70 | RRP9 | FGFBP1 | IVD | CRB1 | TBCCD1 | SLC26A4 | TMCO3 | SOST | KIF9 |
| SLC17A3 | KIF22 | SPRR1B | GORASP2 | GPR37L1 | JUP | SLC26A4 | PYCR1 | SOST | BTD |
| TMEM174 | PACRG | KRT16 | GNB1 | PMP2 | WBP11 | ZBED2 | MPZL1 | SOST | SDSL |
| ASPA | GNL3 | FGFBP1 | THRAP3 | SHISA7 | LTBR | ZBED2 | DNPEP | TBCCD1 | RUFY1 |
| CD70 | KIF22 | DSG3 | CHCHD2 | CRB1 | TES | S100A5 | NUDT2 | ACTL6A | THRAP3 |
| TMEM174 | CCDC151 | IVL | SCCPDH | PMP2 | CDC25B | TMEM233 | RACGAP1 | DCUN1D1 | MXD4 |
| SLC6A13 | RBM15B | DSG3 | THRAP3 | PMP2 | STAT6 | CITED1 | TMCO3 | LSG1 | WIPI2 |
| SLC17A3 | RBM15B | FAM83C | SCCPDH | NCAN | JUP | RXRG | UCK2 | LSG1 | THRAP3 |
| SLC17A3 | GNL3 | SPRR1B | CHCHD2 | SHISA7 | FBXL15 | RXRG | MRPL16 | TBCCD1 | RPS27L |
| SLC6A13 | ZNRF1 | SPRR1B | THRAP3 | NCAN | CDC25B | CITED1 | PYCR1 | GCLC | GDI2 |
| TMEM72 | PYCR1 | TGM1 | TCTA | GFAP | JUP | TMEM233 | UCK2 | KRT74 | ADAM11 |
| MTCP1 | PWWP2B | FAM83C | CHKA | LRRTM3 | TES | SLC26A4 | UCHL5 | DCUN1D1 | DDAH1 |
| TMEM174 | CNKSR1 | GSDMC | CHKA | GFAP | STAT6 | RXRG | UCHL5 | PARL | WIPI2 |
| NAT8 | TXN2 | TGM1 | TMEM39A | NCAN | ZDHHC5 | NKX2-1 | VWA1 | WDR53 | BTD |
| SLC6A13 | ZADH2 | GSDMC | MGAT4A | GFAP | B4GALT1 | MGAT4C | ATAD1 | PARL | MXD4 |
| SLC3A1 | COX7A2L | TGM1 | CRELD1 | SHISA7 | SERINC2 | CITED1 | MRPL16 | ACTL6A | RPS10 |
| SLC3A1 | MAVS | GSDMC | TCTA | PCDH15 | SERINC2 | GABRB2 | ARHGEF9 | TBCCD1 | PGPEP1 |
| SLC3A1 | DYNLRB1 | NIPAL4 | CRELD1 | LRRTM3 | F11R | MGAT4C | UCHL5 | WDR53 | PLEKHA6 |
| NAT8 | FGFRL1 | KRT16 | CHCHD2 | APC2 | WBP11 | S100A5 | EIF4EBP1 | KRT74 | KIF9 |
| NAT8 | COX7A2L | IVL | GORASP2 | PCDH15 | B4GALT1 | NKX2-1 | SP3 | ACTL6A | RNPEP |

| PRAD | | SKCM | | COAD | | STAD | | BLCA | |
|---|---|---|---|---|---|---|---|---|---|
| PRAD_1 | PRAD_2 | SKCM_1 | SKCM_2 | COAD_1 | COAD_2 | STAD_1 | STAD_2 | BLCA_1 | BLCA_2 |
| NKX3-1 | TAGLN2 | MLANA | TOR1AIP1 | NOX1 | ZNF362 | ZFPM1 | B3GAT3 | UPK2 | ALDH7A1 |
| KLK3 | TAGLN2 | MLANA | VOPP1 | CDX2 | PACS1 | ZFPM1 | CD2BP2 | UPK2 | NEO1 |
| KLK3 | LASP1 | MLANA | MYO6 | NOX1 | TCEA2 | ZFPM1 | UROD | UPK2 | ST6GAL1 |
| SLC45A3 | LASP1 | PAX3 | PBX1 | NOX1 | BCAM | ZBTB7A | PRDX5 | PLA2G2F | ALDH2 |
| NKX3-1 | LASP1 | SLC45A2 | DDAH1 | CDX1 | PACS1 | GATA4 | UROD | UPK1A | ST6GAL1 |
| KLK3 | INTS1 | PMEL | TMSB4X | CDX2 | TRIM56 | ZBTB7A | MRFAP1 | UPK1A | HIPK2 |
| ACPP | TAGLN2 | DCT | MYO6 | CDX2 | ZC3H3 | GATA6 | TMEM9 | UPK1A | SH3BP4 |
| ACPP | OGDH | TRPM1 | DDAH1 | GPA33 | PACS1 | GATA4 | TMEM9 | PLA2G2F | STXBP1 |
| ACPP | INTS1 | TRPM1 | PBX1 | GPA33 | ZC3H3 | GATA4 | DNAJB2 | VGLL1 | NFIX |
| SLC45A3 | YWHAH | TRPM1 | RAB3IP | CCL24 | C20orf194 | GNL3L | TSR2 | PLA2G2F | CERK |
| NKX3-1 | KIAA0100 | PMEL | PTPRF | GPA33 | CLU | GATA6 | UBXN6 | SNX31 | CERK |
| SLC45A3 | OGDH | PAX3 | NFYB | CDX1 | BCAM | ZBTB7A | RNF187 | VGLL1 | ST6GAL1 |
| CHRNA2 | TNFAIP8L1 | DCT | VOPP1 | CDX1 | ZC3H3 | ZBTB20 | RNF215 | PPARG | ALDH2 |
| KLK4 | OGDH | PAX3 | VOPP1 | CCL24 | TCEA2 | GATA6 | CD2BP2 | SNX31 | SH3BP4 |
| CHRNA2 | OSBPL3 | DCT | PBX1 | CDH17 | CLU | GNL3L | FN3KRP | SNX31 | OAT |
| OR51E2 | OSBPL3 | SLC45A2 | PAWR | CCL24 | KRBA1 | GNL3L | ADPRHL2 | VGLL1 | OAT |
| CHRNA2 | CIT | SLC45A2 | NET1 | MEPIA | SMARCA1 | CLDN18 | CIRBP | PM20D1 | PARD3B |
| KLK4 | YWHAH | PMEL | NET1 | GUCY2C | BCAM | CLDN18 | PRDX5 | UPK3A | IQGAP2 |
| OR51E2 | TNFAIP8L1 | C10orf90 | DDAH1 | EPS8L3 | CLU | CLDN18 | DNAJB2 | PM20D1 | STXBP1 |
| KLK4 | LAPTM4B | C10orf90 | MAGI1 | GUCY2C | NR3C1 | ZBTB20 | TMED1 | ACER2 | PTPRJ |
| OR51E2 | CIT | C10orf90 | RAB3IP | GUCY2C | MYH10 | NKX6-3 | TMED1 | BTBD16 | CERK |
| SLC30A4 | ANP32E | ALX1 | RAB3IP | MEPIA | TCEA2 | ZBTB20 | MYL6B | UPK3A | COBL |
| HOXB13 | YWHAH | ALX1 | SGMS1 | MEPIA | ABHD8 | NKX6-3 | RNF215 | UPK3B | NFIX |
| SLC30A4 | FAM49B | C19orf71 | SGMS1 | CDH17 | OST4 | CCDC68 | HSDL1 | BTBD16 | COBL |
| HOXB13 | INTS1 | ALX1 | NFYB | PHGR1 | BCL6 | ONECUT2 | DNAJB2 | BTBD16 | RAPGEF5 |
| HOXB13 | KIAA0100 | TYRP1 | SLC38A1 | PHGR1 | ZNF362 | NKX6-3 | HSDL1 | PM20D1 | COBL |
| ANO7 | SERPINB1 | FCRLA | SLC38A1 | PHGR1 | PTPRS | CCDC68 | COQ5 | ACER2 | HIPK2 |
| SLC30A4 | LAPTM4B | TYRP1 | PTPRF | CDH17 | TRIM56 | ONECUT2 | UROD | UPK3B | NFIC |
| ANO7 | S100A16 | TYRP1 | TJP2 | MYO1A | BCL6 | PABPC3 | TMED1 | GRHL3 | KLF13 |
| TRPV6 | S100A16 | TRIM63 | OCIAD2 | NR1I2 | NR3C1 | ONECUT2 | TMEM9 | SNCG | NFIC |
| ANO7 | CDC25B | CAPN3 | SGMS1 | NR1I2 | SMARCA1 | CCDC68 | MYL6B | ACER2 | AGAP1 |
| BEND4 | CIT | C19orf71 | MAGI1 | MYO1A | NR3C1 | ONECUT3 | B3GAT3 | UPK3A | NFIX |
| FOLH1 | LAPTM4B | CAPN3 | MAGI1 | ATOH1 | KRBA1 | MUC13 | PRDX5 | ACOXL | IQGAP2 |
| BEND4 | OSBPL3 | TRIM63 | TJP2 | ATOH1 | LDOC1 | ONECUT3 | MYL6B | GDPD3 | ALDH2 |
| TMEFF2 | FSCN1 | IRF4 | PTPRF | DPEP1 | OBSL1 | ONECUT3 | ADPRHL2 | UPK3B | SH3BP4 |
| BEND4 | FSCN1 | TSPAN10 | TJP2 | PPP1R14D | BCL6 | C6orf222 | APOBR | ACOXL | PTPRJ |
| NWD1 | ANP32E | TRIM63 | PAWR | MYO1A | SMARCA1 | TFF2 | ING4 | PPARG | KLF13 |
| NWD1 | ARHGEF2 | IRF4 | SPINT2 | ISX | TMEM25 | REG4 | B3GAT3 | IL9R | SYBU |
| CHRM1 | FSCN1 | CAPN3 | MYO6 | BCL2L14 | AMOTL1 | REG4 | RNF215 | NIPAL4 | KLF13 |
| TRPV6 | CTSC | IRF4 | SLC38A1 | ASCL2 | PTPRS | REG4 | ING4 | ACOXL | STXBP1 |
| FOLH1 | S100A16 | TSPAN10 | NET1 | BCL2L14 | C20orf194 | CTSE | MRFAP1 | IL9R | IQGAP2 |
| FOLH1 | ANP32E | FOXD3 | NFYB | SLC26A3 | C20orf194 | CTSE | CIRBP | PPARG | GSE1 |
| CHRM1 | CERK | FCRLA | PTPRK | ATOH1 | TMEM25 | MUC5AC | CD2BP2 | IL9R | RASGEF1B |
| ADRB1 | C1GALT1 | ENTHD1 | RBM47 | SLC26A3 | TMEM25 | VSIG1 | ZMAT2 | OR13A1 | SYBU |
| TMEFF2 | ARHGEF2 | TSPAN10 | PSD4 | ISX | LDOC1 | TFF2 | COQ5 | PSCA | NFIC |
| ZNF613 | AGPS | MMP8 | EPCAM | DPEP1 | MYH10 | TFF2 | HSDL1 | GRHL3 | OAT |
| TRPV6 | ARHGEF2 | ENTHD1 | CDS1 | BCL2L14 | PTPRS | MUC5AC | ADPRHL2 | SNCG | PHC2 |
| ZNF613 | CDC25B | FCRLA | PSD4 | ASCL2 | EVL | MUC5AC | GMPR2 | FCRLB | PTPRJ |
| OR51E1 | TNFAIP8L1 | MMP8 | CDS1 | SLC26A3 | KRBA1 | MUC13 | UBXN6 | SNCG | PBXIP1 |
| CHRM1 | CDC25B | EXTL1 | PTPRK | ISX | ABHD8 | CTSE | UBXN6 | GDPD3 | GSE1 |
| LMAN1L | RELT | GPR143 | OCIAD2 | GPR35 | RDX | VSIG1 | ING4 | GDPD3 | HIPK2 |
| ZNF613 | DERA | SNCA | PFN2 | EPS8L3 | TRIM56 | C6orf222 | COQ5 | PSCA | SLC25A23 |
| ADRB1 | RHBDF2 | FOXD3 | SPINT2 | NR1I2 | RDX | PDX1 | APOBR | FCRLB | RASGEF1B |
| ADRB1 | DERA | ENTHD1 | PAWR | GPR35 | EVL | MUC13 | CIRBP | OR13A1 | RASGEF1B |
| STEAP2 | KIAA0100 | MMP8 | RBM47 | PPP1R14D | EVL | VSIG1 | TSR2 | PSCA | GSE1 |
| NWD1 | AGPS | GPR143 | USP39 | PPP1R14D | RDX | C6orf222 | TSR2 | SYT8 | SLC25A23 |
| MSMB | CTSC | GPR143 | SPINT2 | ASCL2 | ZNF362 | TM4SF20 | APOBR | NIPAL4 | RAPGEF5 |
| OR51E1 | RHBDF2 | EXTL1 | BTBD1 | GPR35 | OBSL1 | TM4SF20 | F10 | FCRLB | ALDH7A1 |
| MSMB | SERPINB1 | MMP17 | USP39 | KRT20 | AMOTL1 | PGC | MRFAP1 | TMEM40 | RAPGEF5 |
| LMAN1L | GHRL | FOXD3 | OCIAD2 | KRT20 | TUSC3 | PGC | SNX17 | PADI3 | UTRN |
| DNASE2B | RELT | CA14 | PTPRK | KRT20 | OBSL1 | PGC | ZMAT2 | SYT8 | ALDH7A1 |
| OR51E1 | AGPS | SNCA | BTBD1 | DPEP1 | BNIP3 | TM4SF20 | SLC25A34 | SYT8 | UTRN |
| MSMB | KPNA2 | EXTL1 | USP39 | FAM3D | AMOTL1 | PDX1 | UCK1 | PADI3 | ATXN1 |
| TMEFF2 | CLCN6 | CA14 | CFL2 | VIL1 | OST4 | GJD3 | SLC25A34 | NIPAL4 | ATXN1 |
| POTEH | GHRL | MMP17 | BTBD1 | FAM3D | MYH10 | PDX1 | SLC25A34 | GRHL3 | UTRN |
| DNASE2B | ST6GALNAC4 | SNCA | TOR1AIP1 | EPS8L3 | OST4 | POTEE | PAK6 | TMEM40 | ATXN1 |
| LMAN1L | HS3ST2 | ABCB5 | RBM47 | ATP10B | GALNT1 | GJD3 | TTLL10 | UPK1B | SLC25A23 |
| STEAP2 | TXLNA | CA14 | TOR1AIP1 | ATP10B | FMRI | GJD3 | PAK6 | PADI3 | SMARCA5 |
| POTEH | RELT | MMP17 | CCDC12 | FAM3D | TUSC3 | POTEE | TTLL10 | UPK1B | NEO1 |
| STEAP2 | LIMA1 | ABCB5 | PSD4 | ATP10B | AKIRIN1 | POTEE | LRRC8E | TNNI2 | SYBU |

| LIHC | | CESC | | KIRP | | SARC | | ESCA | |
|---|---|---|---|---|---|---|---|---|---|
| LIHC_1 | LIHC_2 | CESC_1 | CESC_2 | KIRP_1 | KIRP_2 | SARC_1 | SARC_2 | ESCA_1 | ESCA_2 |
| C8B | IGF1R | ARHGEF33 | ZNF608 | LRRN4 | EMP2 | TWIST2 | ERBB3 | ANKRD11 | CD63 |
| SERPINC1 | FAR1 | SYCP2 | INSR | KCP | NOTCH3 | TWIST2 | DSP | ZBTB7A | APH1A |
| C8B | FAR1 | ARHGEF33 | ZNF773 | LRRN4 | TP53I11 | TWIST2 | FAM83H | ANKRD11 | CD81 |
| SERPINC1 | EXOC1 | SYCP2 | TBC1D16 | SMTNL2 | TP53I11 | C1QTNF2 | RAB11FIP4 | ZBTB7A | PEBP1 |
| ASGR2 | MAPRE1 | KCNS1 | PTPRM | LRRN4 | NOTCH3 | FAM180A | ERBB3 | ZBTB7A | PPIB |
| C8B | CTBP2 | CDKN2A | GRINA | TPK1 | UAP1 | RAB23 | TPD52 | EIF3C | NUDT16L1 |
| SERPINC1 | SLC25A36 | ARHGEF33 | ZC4H2 | PKHD1 | NOTCH3 | IL17B | CAMSAP3 | RC3H1 | UFC1 |
| APOC3 | IQGAP1 | SYCP2 | CREB3L2 | LYG1 | TP53I11 | FAM180A | WWC1 | FBRSL1 | PEBP1 |
| ASGR1 | HK1 | KCNS1 | PKIG | SMTNL2 | EMP2 | CCDC36 | CAMSAP3 | FBRSL1 | APH1A |
| KNG1 | HK1 | ZNF541 | PTPRM | SMTNL2 | MFGE8 | CDK15 | ERBB3 | GNL3L | TSR2 |
| CPB2 | HK1 | KCNS1 | PTPRG | TPK1 | ZDHHC20 | C1QTNF2 | PRKCZ | FBXL18 | NUDT16L1 |
| C8A | SLC25A12 | RIBC2 | PKIG | MYL3 | DPYSL3 | SHOX2 | TPD52 | RC3H1 | ANP32A |
| AGXT | FAR1 | EPHX3 | CCND1 | TPK1 | EMP2 | CDK15 | CAMSAP3 | GNL3L | TEX264 |
| AGXT | SLC25A36 | ZNF541 | MOCS1 | LYG1 | NEURL1B | C1QTNF2 | FAM84B | EIF3C | ING4 |
| ASGR1 | TBC1D10B | RIBC2 | ZBTB10 | PTH1R | MFGE8 | FAM180A | RAB11FIP4 | RC3H1 | TMEM9 |
| ASGR2 | PLEKHB2 | ZNF541 | TMEM150A | MYL3 | COL5A3 | TWIST1 | TPD52 | ANKRD11 | MRFAP1 |
| AGXT | ABR | RIBC2 | PTPRM | EMX1 | NEURL1B | MRGPRF | LSR | FBRSL1 | PPIB |
| HAO1 | ZNF827 | SOX30 | ZNF608 | ENAM | COL5A3 | CDK15 | MARVELD2 | HCFC1 | CD81 |
| ASGR1 | ABR | C19orf57 | TBC1D16 | MYL3 | LTBP1 | IL17B | MARVELD2 | NRARP | ANP32A |
| ITIH3 | IQGAP1 | SERPINB3 | CCND1 | KCP | MFGE8 | TWIST1 | F11R | MAPK6 | APH1A |
| C8A | ZNF827 | HMSD | ZNF608 | EMX1 | UAP1 | CCDC36 | MARVELD2 | MAPK6 | PPIB |
| APOC3 | PLEKHB2 | HMSD | ZC4H2 | KCP | MARCKSL1 | TWIST1 | DSP | EIF3C | STK16 |
| APOC3 | CHD3 | TAF7L | ZNF773 | ENAM | NEURL1B | TBXA2R | FAM84B | NRARP | ARF5 |
| APOA5 | ZNF827 | SOX30 | ZC4H2 | SYPL2 | UAP1 | CCDC36 | PRKCZ | GNL3L | PDHB |
| F2 | IQGAP1 | PRDM15 | TBC1D16 | DYNC2LI1 | AZIN1 | TNFAIP8L3 | WWC1 | HCFC1 | CD63 |
| F2 | ARF3 | HMSD | ZNF773 | DYNC2LI1 | SAE1 | TNFAIP8L3 | FAM84B | FBXL18 | ING4 |
| ASGR2 | SLC44A2 | C19orf57 | PKIG | PTH1R | DPYSL3 | IL17B | HOOK1 | KLHL11 | TMED1 |
| F2 | PLEKHB2 | TAF7L | ZNF43 | ENAM | LDLR | MRGPRF | SPINT2 | MAPK6 | MRFAP1 |
| HRG | IGF1R | C19orf57 | FERMT2 | COQ9 | SERP1 | EBF3 | DSP | FBXL18 | STK16 |
| HRG | SLC25A36 | TAF7L | MOCS1 | EMX1 | PCDH1 | MRGPRF | F11R | PABPC3 | TMED1 |
| ITIH2 | CLSTN1 | EPHX3 | CREB3L2 | SYPL2 | PCDH1 | TBXA2R | WWC1 | RBM15 | TSR2 |
| KNG1 | IGF1R | IL20RB | CCND1 | LYG1 | LTBP1 | ADAM33 | MYH14 | ATAD5 | ING4 |
| CPB2 | CTBP2 | CENPK | PTPRG | CYS1 | SERP1 | EBF3 | FAM83H | CLSPN | TSR2 |
| KNG1 | METTL9 | CDC7 | INSR | PTH1R | SAE1 | ADAM33 | LSR | NRARP | CD2BP2 |
| CPB2 | METTL9 | WDR76 | INSR | SULT1C4 | AZIN1 | EBF3 | PRKCZ | KLHL11 | GPANK1 |
| APOH | CLSTN1 | RFC4 | AP2B1 | HOGA1 | SERINC5 | MFAP4 | PTPRF | ZFPM1 | NUDT16L1 |
| C8G | ABR | MEI1 | FERMT2 | HOGA1 | SAE1 | ADAM33 | SPINT2 | RBM15 | PEX11B |
| ITIH3 | CLSTN1 | SERPINB3 | GRINA | DYNC2LI1 | SERP1 | SHOX2 | CXADR | HCFC1 | ILF3 |
| ITIH2 | CCNI | EPHX3 | SNX19 | SLC13A1 | COL5A3 | TNFAIP8L3 | RAB11FIP4 | CLSPN | ELOF1 |
| ITIH2 | ARF3 | SOX30 | PARD3B | SULT1C4 | DPYSL3 | SCARA5 | MYH14 | RBM15 | UROD |
| ITIH3 | CHD3 | LY6K | CREB3L2 | SYPL2 | SERINC5 | RAB23 | PTPRF | ZFPM1 | PEX11B |
| APOH | MAPRE1 | MEI1 | TNS3 | HOGA1 | PCDH1 | LGI2 | LSR | ATAD5 | UROD |
| AMBP | CCNI | SERPINB3 | MTPN | PKHD1 | AZIN1 | SHOX2 | MYH14 | FAM83B | ZMAT2 |
| APOH | ARF3 | MEI1 | SIAE | CYS1 | MARCKSL1 | PTGFR | MAL2 | CLSPN | UROD |
| HAO1 | SLC25A12 | IL20RB | GRINA | SLC13A1 | LTBP1 | HSPB6 | PTPRF | ZFPM1 | STK16 |
| SERPINA10 | METTL9 | PSMC3IP | TMEM150A | CYS1 | BAZ2A | LGI2 | CXADR | ATAD5 | PEX11B |
| HRG | CTBP2 | LY6K | ZBTB10 | SULT1C4 | MARCKSL1 | LGI2 | SPINT2 | FAM83B | DNAJB2 |
| SERPINA10 | CHMP3 | CDC7 | PTPRG | PKHD1 | BAZ2A | SCARA5 | MAL2 | FAM83B | ANP32A |
| C8G | SLC44A2 | WDR76 | SNX19 | SLC17A1 | SERINC5 | PTGFR | CXADR | REL | PDHB |
| C8G | DCTN5 | CDKN2A | TNS3 | SLC13A1 | PRRX1 | PTGFR | CDH1 | REL | TEX264 |
| SERPINA10 | PRKRA | GPR87 | SNX19 | SLC17A1 | LDLR | RAB23 | RNF11 | REL | TMEM9 |
| APOC2 | SLC25A12 | LY6K | FERMT2 | SLC17A1 | SLC22A23 | PTX3 | MAL2 | PABPC3 | GPANK1 |
| C8A | MTMR2 | CDKN2A | AP2B1 | SLCO4C1 | ZDHHC20 | TBXA2R | CDH1 | TMPPE | TMED1 |
| AHSG | DCTN5 | WDR76 | TNS3 | PAX2 | ZDHHC20 | SCARA5 | FAM83H | MXD1 | ARF5 |
| APOA2 | CCNI | CENPK | SIAE | SLCO4C1 | BAZ2A | EBF1 | F11R | MXD1 | MRFAP1 |
| AHSG | CHD3 | CENPK | ZBTB10 | MIOX | TSPAN13 | EBF1 | CTSO | MXD1 | PARK7 |
| AHSG | SLC44A2 | IL20RB | AP2B1 | SLC3A1 | LDLR | PTX3 | HOOK1 | GJD3 | SOWAHA |
| HAO1 | MTMR2 | S1PR5 | SIAE | SLCO4C1 | TSPAN13 | PTX3 | CDH1 | TMPPE | GPANK1 |
| APOC2 | MTMR2 | GPR87 | MARVELD1 | PAX2 | TSPAN13 | SYDE1 | KRT18 | GJD3 | ATRIP |
| APOA5 | C6orf203 | PSMC3IP | MOCS1 | MIOX | SQLE | HSPA12B | DDX54 | PABPC3 | SLC11A1 |
| APOA5 | EFCAB2 | KLHDC7B | TMEM150A | MIOX | INTS7 | HSPB6 | KRT18 | GJD3 | NLRP14 |
| APOA2 | MAPRE1 | KLHDC7B | MARVELD1 | PAX2 | BCL6 | EBF1 | RNF11 | POTEE | ATRIP |
| APOC2 | PRKRA | CDC7 | CRY1 | CDH16 | PIH1D1 | HSPA12B | UBN1 | KLHL11 | ZFYVE28 |
| VTN | WBP2 | GPR87 | PRKCD | SLC3A1 | SQLE | MFAP4 | KRT18 | POTEE | SOWAHA |
| APOA2 | DCTN5 | KLHDC7B | PRKCD | SLC3A1 | PIH1D1 | HSPA12B | MAP3K7 | PLEC | CD63 |
| AMBP | WBP2 | S1PR5 | ZNF43 | CDH16 | SQLE | MFAP4 | KRT8 | POTEE | WNT16 |
| ALB | WBP2 | PSMC3IP | EPDR1 | CDH16 | MTHFD2 | SYDE1 | KRT8 | PLEC | CD81 |
| VTN | CHMP3 | S1PR5 | EPDR1 | GLYAT | BCL6 | HSPB6 | KRT8 | PLEC | PEBP1 |
| VTN | PRMT2 | RFC4 | FOXJ3 | GLYAT | SLC22A23 | SYDE1 | SPINT1 | TMPPE | ZFYVE28 |
| AMBP | PRMT2 | CENPW | PRKCD | GLYAT | ITGAL | KANK2 | SPINT1 | C11orf91 | NLRP14 |

| PAAD | | PCPG | | READ | | TCGT | | THYM_1 | |
|---|---|---|---|---|---|---|---|---|---|
| PAAD_1 | PAAD_2 | PCPG_1 | PCPG_2 | READ_1 | READ_2 | TGCT_1 | TGCT_2 | THYM_1 | THYM_2 |
| GCG | FOXRED2 | CHRNA3 | YBX1 | LY6G6D | SNX24 | VRTN | MFSD6 | PAX1 | DSTN |
| GCG | ORC3 | SLC18A1 | TMEM63A | CDX2 | DTX3L | LIN28A | EFNA1 | PRSS16 | NCKAP1 |
| GCG | MCUR1 | CHRNA3 | SERBP1 | CDX2 | NFIC | LIN28A | CHMP3 | PRSS16 | DSTN |
| CPA1 | FOXRED2 | PHOX2A | LSR | LY6G6D | KRBA1 | VRTN | TICAM1 | PAX1 | NCKAP1 |
| CPA1 | MCUR1 | CHRNA3 | IDH2 | LY6G6D | KCTD1 | LIN28A | ELOVL1 | FOXN1 | DHCR24 |
| CPA1 | TMEM69 | TH | ERBB2 | NOX1 | GPD2 | VRTN | MBNL2 | PRSS16 | CALU |
| G6PC2 | KCNAB1 | TH | YBX1 | NOX1 | SS18 | DPPA4 | EXOC3 | PAX1 | CALU |
| CLPS | MMACHC | TH | ANXA11 | NOX1 | STOM | DPPA4 | KLHDC10 | RAG1 | ZDHHC9 |
| CLPS | SUV39H2 | PHOX2A | NOTCH2 | CDX2 | STOM | TRIM71 | IRF2BP2 | CHRM4 | CAMK2N1 |
| CLPS | RFC5 | DBH | KIF1C | CCL24 | RNF144B | TRIM71 | PGRMC1 | GRAP2 | DHCR24 |
| G6PC2 | L2HGDH | DRD2 | IDH2 | GPA33 | NFIC | DPPA4 | COMT | CCR9 | CAMK2N1 |
| CPA2 | FOXRED2 | DBH | IDH2 | CCL24 | C20orf194 | GDF3 | TICAM1 | SLC46A2 | EPS8 |
| CASR | L2HGDH | DBH | ZFP36L1 | GPR35 | NFIC | GDF3 | AIG1 | RAG1 | DHCR24 |
| G6PC2 | SUV39H2 | HAND2 | YBX1 | GPA33 | STOM | GDF3 | EFNA1 | FOXN1 | NCKAP1 |
| CPA2 | RFC5 | SLC18A1 | TRAF4 | AIFM3 | EVL | TRIM71 | PHC2 | RAG1 | SLC31A1 |
| CASR | CLPB | PHOX2A | ERBB2 | GPA33 | DTX3L | POU5F1 | TMEM59 | PTCRA | PCDH1 |
| CASR | CELSR2 | SLC18A1 | PTGFRN | CCL24 | KCTD1 | POU5F1 | DAZAP2 | PTCRA | SOX13 |
| CPA2 | TMEM69 | HAND2 | ZFP36L1 | AIFM3 | BCL6 | POU5F1 | CAST | FOXN1 | BAG3 |
| CHST4 | RFC5 | MAB21L1 | NOTCH2 | RXFP4 | KRBA1 | FOXH1 | EFNA1 | LAT | CAMK2N1 |
| PNLIPRP2 | ARMC6 | DRD2 | PTGFRN | SLC26A3 | NR3C1 | TRIML2 | KDSR | SLC46A2 | PCDH1 |
| PLA2G1B | PCCB | MAB21L1 | REST | CDX1 | SS18 | TRIML2 | TICAM1 | PTCRA | ZDHHC9 |
| CHST4 | MMACHC | MAB21L1 | TRAF4 | ASCL2 | SS18 | TRIML2 | FBXO3 | GRAP2 | ZDHHC9 |
| PLA2G1B | ATPAF1 | DGKK | NOTCH2 | PPP1R14D | NR3C1 | ZSCAN10 | AIG1 | GRAP2 | BAG3 |
| PNLIPRP2 | PCCB | PENK | ZFP36L1 | SLC26A3 | KCTD1 | VENTX | PPA2 | CCR9 | SOX13 |
| PNLIPRP2 | TMEM209 | HAND2 | SERBP1 | PPP1R14D | BCL6 | FOXH1 | MBNL2 | CHRM4 | EPS8 |
| PLA2G1B | BTBD6 | TLX2 | RCC1 | ISX | NR3C1 | VENTX | CHMP3 | CD3D | EFHD2 |
| CHST4 | CLPB | TLX2 | TMEM63A | SLC26A3 | RAB12 | L1TD1 | CAST | UBASH3A | BAG3 |
| CUZD1 | CLPB | TLX2 | REST | CDX1 | PTPRS | L1TD1 | TMEM59 | CCR9 | MANSC1 |
| CUZD1 | TMEM209 | DRD2 | ERBB2 | ISX | SMARCA1 | ZFP42 | ELOVL1 | APOBEC2 | PCDH1 |
| SLC30A8 | CELSR2 | INSM2 | LRRC1 | CDX1 | SART1 | SLC2A14 | AIG1 | MEIG1 | MANSC1 |
| CUZD1 | ORC3 | DRGX | RCC1 | PPP1R14D | TANC2 | VENTX | ELOVL1 | TRAT1 | FAM114A1 |
| SCTR | SOX12 | DRGX | RPS6KA1 | MEPIA | WWTR1 | FOXH1 | MFSD6 | CD3D | JTB |
| FOXL1 | BTBD6 | DRGX | NEK6 | MEPIA | BCL6 | HYAL4 | MFSD6 | ZAP70 | EFHD2 |
| SCTR | BTBD6 | SLC18A2 | VAMP8 | GUCY2C | WWTR1 | SLC2A14 | KLHDC10 | SH2D1A | PLBD2 |
| GPBAR1 | SUV39H2 | SLC18A2 | NEK6 | ASCL2 | EVL | ZFP42 | PTPRK | SLC46A2 | MANSC1 |
| SCTR | MCUR1 | NEUROD4 | LRRC1 | MEP1A | EVL | ZFP42 | MBNL2 | SH2D1A | CALU |
| SFRP5 | CELSR2 | SLC18A2 | TMEM63A | AIFM3 | RAB12 | ZSCAN10 | PTPRK | CCL25 | DSTN |
| GPBAR1 | MMACHC | TBX20 | LRRC1 | MYO1A | WWTR1 | L1TD1 | DAZAP2 | SH2D1A | DUSP3 |
| SFRP5 | SOX12 | DGKK | TRAF4 | GUCY2C | RDX | SLC2A14 | ZADH2 | CD3G | ADAM9 |
| FOXL1 | PPIL1 | INSM2 | NEK6 | DPEP1 | MYH10 | HYAL4 | ZADH2 | UBASH3A | CDC42EP1 |
| TFF2 | PPIL1 | PENK | SERBP1 | ISX | C20orf194 | HYAL4 | FBXO3 | CD3G | PTK2 |
| SLC30A8 | SOX12 | CHGB | B2M | R3HDML | KRBA1 | ZSCAN10 | ZADH2 | CHRM4 | SOX13 |
| SFRP5 | ATPAF1 | DGKK | TSPAN6 | ASCL2 | SART1 | DPPA2 | PTPRK | UBASH3A | FAM114A1 |
| TFF2 | TMEM69 | NEUROD4 | TSPAN6 | DPEP1 | ECH1 | SLC7A3 | NFIC | SIT1 | CDC42EP1 |
| FOXL1 | ARMC6 | NEUROD4 | RCC1 | GUCY2C | CDC23 | SLC7A3 | KLHDC10 | APOBEC2 | CDC42EP1 |
| TFF2 | PCCB | FAM163A | ANXA11 | CDH17 | ZFP36 | SLC7A3 | KDSR | SIT1 | B4GALT2 |
| SLC30A8 | TMEM209 | HAND1 | CDH1 | NR1I2 | SMARCA1 | NODAL | SETD7 | ZAP70 | PLBD2 |
| GLP2R | L2HGDH | RTL1 | YAP1 | PHGR1 | PTPRS | NANOS3 | EXOC3 | CD3G | DUSP3 |
| REG1B | CSE1L | RTL1 | TGIF1 | PHGR1 | RNF144B | NANOS3 | PPA2 | CD247 | PLBD2 |
| REG1B | GLO1 | PENK | SF3B2 | PHGR1 | RAB12 | NANOS3 | CHMP3 | ZAP70 | JTB |
| REG1B | MTCH2 | VWA5B2 | ANXA11 | DPEP1 | RDX | CLEC4D | SETD7 | SLAMF1 | DUSP3 |
| TM4SF4 | ATPAF1 | RTL1 | LSR | CDH17 | DTX3L | NLRP9 | SETD7 | TRAT1 | SLC31A1 |
| CFC1 | GNMT | TBX20 | STXBP2 | CDH17 | ECH1 | OOEP | FBXO3 | CCL25 | ERBB3 |
| TM4SF4 | ARMC6 | SLC6A2 | LSR | GUCA2A | TMEM25 | NLRP9 | LRRCC1 | CD247 | CD276 |
| TM4SF4 | TRUB2 | SLC6A2 | VAMP8 | RXFP4 | CLIP4 | NLRP9 | PPA2 | APOBEC2 | FAM114A1 |
| ANXA10 | PPIL1 | KCNG4 | STXBP2 | NR1I2 | GNB5 | RNF17 | KDSR | CCL25 | EFHD2 |
| ANXA10 | TRUB2 | HAND1 | REST | GPR35 | NAGA | RNF17 | PGRMC1 | CD3D | YARS |
| RBPJL | METTL4 | INSM2 | TSPAN6 | NR1I2 | RDX | DPPA2 | IL13RA1 | SLAMF1 | ADAM9 |
| RBPJL | SNRNP25 | SLC6A2 | KIF1C | MYO1A | GNB5 | RNF17 | EXOC3 | TRAT1 | EPS8 |
| RBPJL | PCBD2 | CHGA | B2M | MYO1A | SMARCA1 | CLEC4D | LRRCC1 | SLAMF1 | SLC31A1 |
| ANXA10 | SNRNP25 | FAM163A | KIF1C | EPS8L3 | ZFP36 | CLEC4D | RPIA | CD8B | CD276 |
| FFAR1 | GNMT | HAND1 | STXBP2 | GUCA2A | C20orf194 | DPPA2 | PGRMC1 | CD8B | JTB |
| FFAR1 | KCNAB1 | TBX20 | CDH1 | GUCA2A | B3GALNT1 | NODAL | LRRCC1 | CD247 | PTK2 |
| FFAR1 | PCBD2 | KCNG4 | CDH1 | FAM3D | PTPRS | NODAL | RPIA | SIT1 | PRKAR2A |
| C1orf127 | PCBD2 | FAM163A | PLIN3 | GPR35 | MYH10 | OOEP | RPIA | CD8B | PTK2 |
| C1orf127 | GPN3 | KCNG4 | PHF7 | RXFP4 | B3GALNT1 | OOEP | IL13RA1 | LAT | CCDC142 |
| CFC1 | PLCXD2 | VWA5B2 | DBNL | FAM3D | ZNF532 | RPL10L | RHOF | TTC24 | CCDC142 |
| GLP2R | KCNAB1 | CARTPT | YAP1 | EPS8L3 | NAGA | ZNF99 | IL13RA1 | TTC24 | WWC1 |
| GPBAR1 | GPN3 | VWA5B2 | PTGFRN | FAM3D | GPD2 | HOXB1 | RHOF | MEIG1 | WWC1 |
| C1orf127 | SNRNP25 | CARTPT | RPS6KA1 | EPS8L3 | MYH10 | HOXB1 | MATN3 | LAT | ADAM9 |

TABLE 2
Gene Pairs For UCEC Sub-Types

| Solid Tissue | Solid Tissue | | | | |
|---|---|---|---|---|---|
| Normal_1 | Normal_2 | Endometrioid_1 | Endometrioid_2 | Serous_1 | Serous_2 |
| RERG | MKI67 | FOXA2 | MAGEH1 | L1CAM | CDKN1A |
| RERG | TMEM132A | KIAA1324 | NPR1 | L1CAM | MOB3A |
| SLC22A3 | MYBL2 | SPDEF | NPR1 | L1CAM | NFIC |
| PLSCR4 | ZDHHC16 | SPDEF | HIF3A | CLDN6 | CDKN1A |
| PLSCR4 | NUP43 | FOXA2 | HIF3A | CLDN6 | MOB3A |
| TCF23 | MYBL2 | FOXA2 | PNMA3 | CLDN6 | NFIC |
| MAMDC2 | MYBL2 | NANS | NPR1 | GRB7 | CDKN1A |
| GATA6 | TK1 | SPDEF | MAGEH1 | GRB7 | MOB3A |
| PLSCR4 | FTSJ1 | MYBL2 | L1CAM | PNMA3 | IL20RA |
| RSPO1 | MKI67 | BSPRY | L1CAM | MYBL2 | KIAA1324 |
| BCHE | MKI67 | KIAA1324 | HIF3A | SLC6A12 | IL20RA |
| SLC22A3 | CDC20 | NANS | ARHGAP23 | CDC20 | KIAA1324 |
| RERG | TK1 | GALNT10 | ARHGAP23 | GPRIN2 | IL20RA |
| GATA6 | CDC20 | CDC20 | L1CAM | UNK | KIAA1324 |
| RSPO1 | CDC20 | KIAA1324 | FBXO17 | GRB7 | PGR |
| RSPO1 | TK1 | BSPRY | SLC6A12 | PNMA3 | PGR |
| GATA6 | ZDHHC16 | OSTF1 | FBXO17 | SLC6A12 | PGR |
| MAGEH1 | FTSJ1 | BSPRY | FAM110B | CTCFL | NIPAL1 |
| ASPA | EME1 | MLPH | ARHGAP23 | SLC6A12 | PXK |
| BCHE | TBC1D7 | OSTF1 | MAGEH1 | TBC1D7 | SPDEF |

TABLE 3
Gene Pairs For STAD Sub-Types

| Intestinal_1 | Intestinal_2 | Diffuse_1 | Diffuse _2 |
|---|---|---|---|
| HOOK1 | JAM2 | ABCA8 | SHPRH |
| BUB1 | OGN | CHRDL1 | TNIK |
| HOOK1 | CHRDL1 | OGN | VPS37A |
| HOOK1 | OGN | NGFR | LYRM4 |
| FAM136A | GYPC | JAM2 | LYRM4 |
| AURKA | OGN | CHRDL1 | TRAFD1 |
| BUB1 | NGFR | JAM2 | STIM2 |
| DSN1 | JAM2 | JAM2 | VPS37A |
| BUB1 | JAM2 | NGFR | SHPRH |
| DSN1 | SELP | CADM3 | ZNF112 |
| DSN1 | ABCA8 | SRPX | STIM2 |
| PIGU | GYPC | ABCA8 | LYRM4 |
| RAE1 | BOC | CHRDL1 | VPS37A |
| AURKA | NGFR | OGN | TRAFD1 |
| UBE2C | GYPC | PKNOX2 | ZNF112 |

TABLE 4
Gene Pairs For PADD Sub-Types

| LowPurity_1 | LowPurity_2 | basal_1 | basal_2 | classical_1 | classical_2 |
|---|---|---|---|---|---|
| RHOJ | EFNA4 | BCAR3 | BTG2 | LRRC66 | LDLRAD3 |
| JAM2 | SAMD10 | GPR87 | FRZB | IHH | DSE |
| PREX1 | PTK6 | COX6B2 | NOSTRIN | LRRC66 | TTC7B |
| FBLN5 | MANBAL | FBXL2 | FRZB | ZFPM1 | RDX |
| CYYR1 | EFNA4 | COX6B2 | FMO5 | IHH | CAMK1D |
| ERG | EFNA4 | BEAN1 | NOSTRIN | SPIRE2 | CHST11 |
| FBLN5 | ICA1 | MET | CAPRIN1 | FMO5 | PTPRS |
| CXCL12 | KRTCAP3 | GPR87 | NOSTRIN | FMO5 | MYO5A |
| ST8SIA4 | SAMD10 | RYK | BTG2 | TM4SF5 | CAMK1D |
| BCL2 | SAMD10 | GPR87 | FMO5 | C9orf152 | CITED2 |
| SAMHD1 | MST1R | COX6B2 | BLNK | TM4SF5 | PTPRS |
| FBLN5 | ELMO3 | NT5E | BTG2 | C9orf152 | PTPRS |
| SAMHD1 | B3GNT3 | BCAR3 | TMEM98 | IHH | MYO5A |
| MPP1 | SPIRE2 | BEAN1 | KALRN | TM4SF5 | MCC |
| JAM2 | NXT1 | FBXL2 | RAI2 | C9orf152 | PHLDB2 |
| BCL2 | PORCN | FBXL2 | PDX1 | SPIRE2 | FMNL1 |
| PRCP | OCIAD2 | ANXA8 | ARHGAP24 | AGR3 | EVL |
| PRCP | SSH3 | ANXA8 | RAI2 | SPIRE2 | RDX |
| PRCP | B3GNT3 | SIX4 | CHN2 | ZFPM1 | FMNL1 |
| GNG2 | NXT1 | NT5E | TMEM98 | LRRC66 | SACS |
| GIMAP4 | IGSF9 | BEAN1 | PDX1 | ZFPM1 | CHST3 |
| RASSF2 | ADAP1 | ANXA8 | BLNK | ANKS4B | CAMK1D |
| ADPRH | C1D | TNNT1 | EXOC6 | AGR3 | RDX |
| CELF2 | PITX1 | ARNTL2 | MAPRE2 | AGR3 | DENND5A |
| BCL2 | C1D | PORCN | KALRN | FMO5 | PHLDB2 |
| JAM2 | IGSF9 | BCAR3 | MAPRE2 | FOXA3 | EFEMP1 |
| SAMHD1 | OCIAD2 | TNNT1 | KALRN | TRIM15 | PHLDB2 |
| CYYR1 | IGSF9 | PORCN | C1orf115 | FOXA3 | NDST1 |
| METTL7A | TSPAN15 | ADAMTSL5 | FMO5 | TRIM15 | CHST3 |
| ST8SIA4 | C1D | SIX4 | ASRGL1 | NPAS1 | P2RY6 |
| GIMAP4 | PITX1 | PTK6 | ATP2A3 | ICA1 | ELL2 |
| CD8A | ADAMTSL5 | PORCN | ARL15 | KALRN | EVL |
| CD8A | CENPE | PLXNA1 | CTSS | ADAP1 | DNAJC13 |
| CERKL | CENPE | PLXNA1 | ATP2A3 | CRB3 | NIN |
| ST8SIA4 | PORCN | FSCN1 | ATP2A3 | ANKS4B | DYSF |
| ERG | NXT1 | TNNT1 | PDX1 | ADAP1 | EVL |
| RASSF2 | PTK6 | SIX4 | ARHGAP24 | USH1C | CNN3 |
| CXCL12 | SH3RF1 | C16orf74 | CEBPA | ADAP1 | CHST11 |
| CXCL12 | PREB | MET | CTSS | LRCH1 | DENND5A |
| PREX1 | ICA1 | FAM83A | METTL7A | KALRN | NIN |
| RHOJ | SPIRE2 | ARNTL2 | IQGAP2 | BDH1 | DYSF |
| AOAH | ADAMTSL5 | PTK6 | EPS8L3 | USH1C | ETS1 |
| GAB3 | ADAMTSL5 | C16orf74 | ASRGL1 | APOBEC1 | P2RY6 |
| MPP1 | PITX1 | SNCG | LPAR6 | TRIM15 | DYSF |
| PREX1 | ADAP1 | SNCG | C1orf115 | FOXA3 | FMNL1 |
| CD8A | CHEK2 | PTK6 | IQGAP2 | EPS8L3 | ETS1 |
| EVL | PREB | SNCG | ARL15 | SLC45A3 | NDST1 |
| GIMAP6 | CENPV | PRRC1 | METTL7A | TJP3 | ETS1 |
| GIMAP4 | VAMP4 | FAM3C | METTL7A | CYP251 | CNN3 |
| GIMAP8 | RBFA | ITGA3 | R13516 | ITPKA | SLC37A2 |

TABLE 5
Gene Pairs For LUSC Sub-Types

| primitive_1 | primitive_2 | secretory_1 | secretory_2 | basal_1 | basal_2 | classical_1 | classical_2 |
|---|---|---|---|---|---|---|---|
| SBK1 | MAFB | CIITA | PIR | SERPINB3 | TXNRD1 | TMEM116 | GPSM3 |
| ATAT1 | IL1RN | FMNL1 | FBXO45 | HES2 | MEGF9 | MRAP2 | ACSL5 |
| MEX3A | MAFB | TNFRSF1B | SIAH2 | IL1RN | TXNRD1 | CYP4F3 | KRT7 |
| CSTF1 | RIN2 | TNFRSF1B | POLR2H | CXCL1 | CDK5RAP2 | TSPAN7 | FAM107B |
| SBK1 | IL1RN | TNFRSF1B | ZNF639 | SERPINB3 | EPCAM | TMEM116 | ZFAND2B |
| SBK1 | S100A8 | RFTN1 | FBXO45 | FAM83A | CDK5RAP2 | MRAP2 | PDZD2 |
| FAM184A | RAB27B | FMNL1 | MRPL47 | CXCL1 | RIT1 | OSGIN1 | CXXC5 |
| FAM184A | CIITA | ABI3BP | ECE2 | PTPRH | FANCC | OSGIN1 | CRIP2 |
| HES6 | MAFB | ANXA6 | ACTL6A | PTK6 | MAFG | TMEM116 | CXXC5 |
| HES6 | S100A8 | FLI1 | DENND2C | CXCL1 | ME1 | ME1 | PHC2 |
| FAM184A | ABI3BP | SELPLG | ECE2 | PTK6 | CDK5RAP2 | ADAM23 | PHC2 |
| TOX3 | TMEM116 | ANXA6 | PCYT1A | FABP5 | STARD7 | MRAP2 | TMEM51 |
| VIL1 | SERPINB3 | ANXA6 | GMPS | FAM83A | GTF3C4 | MAFG | FAM107B |
| HES6 | GJB3 | BIRC3 | ZNF639 | GPR153 | CTNNAL1 | CYP4F11 | CRIP2 |
| MEX3A | PHLDA3 | ETS1 | PCYT1A | GPR153 | GTF3C4 | TSPAN7 | PMEPA1 |
| SRCIN1 | ANXA8 | TGM2 | PFN2 | FAM83A | MAFG | TSPAN7 | CRIP2 |
| MEX3A | TUBB6 | ABI3BP | MOB2 | FABP5 | TXNRD1 | SCN9A | CXXC5 |
| TUBB2B | RAC2 | ABI3BP | DENND2C | SERPINB3 | ME1 | SCN9A | SLC43A3 |
| VIL1 | S100A8 | C1orf162 | DENND2C | CXCL6 | WASF1 | SCN9A | GPSM3 |
| SRCIN1 | RAB27B | FLI1 | WDR53 | S100A8 | TALDO1 | CYP4F11 | PHC2 |
| VIL1 | ANXA8 | SLCO2A1 | PIR | GJB3 | CBX1 | CYP4F11 | KRT7 |
| ATAT1 | RAB27B | CIITA | MAFG | FABP5 | PGD | PIR | TRIM8 |
| TUBB2B | TNFRSF1B | LTB | GPX2 | EPS8L1 | CTNNAL1 | ME1 | PTP4A2 |
| TOX3 | PDZK1IP1 | TSPAN4 | FBXO45 | HES2 | GTF3C4 | OSGIN1 | TMEM51 |
| ATAT1 | GJB3 | BIRC3 | RIT1 | HES2 | MAFG | TXN | SDC4 |

TABLE 6
Gene Pairs For LUAD Sub-Types

| prox.-inflam_1 | prox.-inflam_2 | TRU_1 | TRU_2 | prox.-prolif._1 | prox.-prolif._2 |
|---|---|---|---|---|---|
| CD274 | KIAA1324 | PLA2G4F | NUF2 | CABYR | PER3 |
| BEND6 | GJB1 | SCTR | CEP55 | FGL1 | PER3 |
| TNFSF4 | GJB1 | SCTR | KIF2C | C2CD4D | HPGDS |
| SPHK1 | C9orf152 | SCTR | KIF4A | FGL1 | TLR2 |
| RGS10 | RAP1GAP | PLA2G4F | NEK2 | FGL1 | CIITA |
| PLAU | MTUS1 | PLLP | BIRC5 | CABYR | ARHGAP20 |
| NTAN1 | FAM174B | PLA2G4F | PRR11 | SLC16A14 | CIITA |
| PDCD1LG2 | GJB1 | HLF | KIF11 | CABYR | MAML2 |
| DSE | RAP1GAP | PLLP | CDK1 | SLC16A14 | MAML2 |
| CMTM3 | RAP1GAP | HLF | CEP55 | VAX2 | HPGDS |
| ANLN | GPT2 | SUSD2 | KPNA2 | FGA | DPYD |
| CTHRC1 | CIT | INMT | BIRC5 | FGA | HLA-DMB |
| ANLN | CABLES1 | ADAMTS8 | CENPA | SLC48A1 | TLR2 |
| CD274 | INMT | HLF | BUB1 | SLC16A14 | ATP10A |
| TPX2 | GPT2 | ADAMTS8 | PBK | ABCB6 | FAS |
| RGS10 | FAM174B | ADAMTS8 | NUF2 | GPT2 | EMP1 |
| DSE | CABLES1 | INMT | KIF11 | FGA | CIITA |
| NTAN1 | KIAA1324 | TNXB | KIF11 | GPT2 | HLA-DMB |
| DSE | SLC48A1 | SCN4B | CKAP2L | PBK | ATP10A |
| CD109 | TOB1 | INMT | CDK1 | ENO3 | ARHGAP20 |
| CD109 | FAM174B | RTN4RL1 | CENPA | S100P | EMP1 |
| RGS10 | SLC48A1 | TMPRSS2 | KPNA2 | PBK | DAPP1 |
| CD109 | KIAA1324 | SCN4B | CENPA | ENO3 | PER3 |
| CD274 | C9orf152 | CBX7 | CEP55 | PBK | FAS |
| ANLN | SORBS2 | NFIX | KPNA2 | GPT2 | SPRED1 |

TABLE 7
Gene Pairs For LGG Sub-Types

| ME_1 | ME_2 | PN_1 | PN_2 | CL_1 | CL_2 | NE_1 | NE_2 |
|---|---|---|---|---|---|---|---|
| IL1R1 | KLHL23 | SLCO5A1 | NIPAL2 | MEOX2 | NALCN | NAPB | LIMA1 |
| IL1R1 | BCL7A | FERMT1 | KCNAB2 | IGFBP2 | ACTR1A | NAPB | MIDN |
| IL1R1 | DSCAM | DSCAM | SYNPO | MEOX2 | REPS2 | CAMKK1 | NKIRAS2 |
| TYMP | CRTC1 | FAM110B | SYNPO | MEOX2 | GNAI1 | GDA | NKIRAS2 |
| TYMP | BCL7A | FERMT1 | SYNPO | TLK1 | RAB18 | MAL2 | NUBP1 |
| TYMP | RUNDC3A | SHD | NAPB | FBXO17 | TMEFF2 | KCNAB2 | MIDN |
| CD3D | TBR1 | GPR173 | UGP2 | HS3ST3B1 | PCBP3 | KCNAB2 | LIMA1 |
| GPR65 | ANAPC1 | SLCO5A1 | OCIAD2 | PIPOX | MAGEH1 | KCNAB2 | CDC42SE1 |
| RAB27A | MEIS1 | BCL7A | UGP2 | PIPOX | DNM3 | SULT4A1 | PPP1R18 |
| GPR65 | PTS | SLCO5A1 | RGS14 | SHOX2 | H2AFY2 | SULT4A1 | LIMA1 |
| MYO1G | EDN3 | PCGF2 | FAM131A | HS3ST3B1 | H2AFY2 | SV2B | NUP188 |
| TNFAIP8 | EDN3 | SHD | KCNAB2 | MEIS1 | GNAI1 | GDA | WDR81 |
| RAB27A | ANAPC1 | SHD | UGP2 | MEIS1 | ASB13 | SULT4A1 | NUP188 |
| GPRC5A | RCOR2 | FERMT1 | SIPA1L1 | SH2D4A | PCBP3 | CAMKK1 | TRAFD1 |
| FAM20A | KLHL23 | DSCAM | SIPA1L1 | OCIAD2 | TMEFF2 | SV2B | PPP1R18 |
| CD3D | GABRA1 | RCOR2 | FAM131A | SHOX2 | PCBP3 | GABRA1 | NKIRAS2 |
| RAB27A | KLHL23 | RCOR2 | RALB | PIPOX | ARL3 | CACNG3 | DDX19B |
| KYNU | EDN3 | GPR173 | HOPX | HS3ST3B1 | TMEFF2 | SYNPR | BAZ1A |
| CD3G | TBR1 | GPR173 | FAM131A | IGFBP2 | WAC | RBFOX1 | BAZ1A |
| CD96 | CACNG3 | BCL7A | SIPA1L1 | IGFBP2 | SAR1A | MAL2 | ANAPC1 |
| PTPN22 | CACNG3 | JPH4 | NAPB | FBXO17 | GNAI1 | TBR1 | DDX19B |
| PTPN22 | RYR2 | H2AFY2 | CAMKK1 | DMRTA2 | AIFM2 | NAPB | PTBP1 |
| CD96 | TBR1 | DSCAM | HOPX | MCCC1 | ARL3 | CAMKK1 | ARHGAP17 |
| TNFAIP8 | AIFM2 | ZNF74 | CYB561 | MEIS1 | GALNT13 | PTER | DDX19B |
| GPRC5A | CAMKK1 | USP49 | CYB561 | FBXO17 | REPS2 | PTER | NUBP1 |
| TREM1 | SYNPR | TMEFF2 | CAMKK1 | DMRTA2 | DDX19B | GDA | STK10 |
| GPRC5A | ZNF74 | RCOR2 | HOPX | DMRTA2 | TTN | SV2B | TRAFD1 |
| MYO1G | AMY2B | PCGF2 | RALB | MCCC1 | DNM3 | PTER | INTS9 |
| FAM20A | ZNF74 | USP49 | CXCL14 | ARAP3 | DNM3 | RYR2 | BAZ1A |
| FAM20A | DSCAM | ZNF74 | LGALS8 | SHOX2 | TTN | CCK | STK10 |
| CD3D | RBP4 | JPH4 | KCNAB2 | NPNT | JPH4 | CPNE6 | MAN2B1 |
| CD96 | MAL2 | USP49 | DYNLT3 | ARAP3 | GALNT13 | CACNG3 | NUBP1 |
| GPR65 | MEIS1 | ZNF74 | DYNLT3 | SHROOM3 | REPS2 | RBFOX1 | STK10 |
| SNX20 | AIFM2 | GALNT13 | NAPB | OTX1 | SH3GL2 | CACNG3 | ANAPC1 |
| TREM1 | GABRA1 | PTS | KLHL26 | PDPN | JPH4 | CPNE6 | WDR81 |
| TREM1 | RYR2 | KLHL23 | RALB | TNFAIP6 | H2AFY2 | RBFOX1 | MAN2B1 |
| CD3G | SH2D7 | PCGF2 | CXCL14 | WIPF3 | SH3GL2 | FAM131A | TRAFD1 |
| PTPN22 | HCN1 | AMOTL2 | ANKRD11 | PDPN | MXI1 | SYNPR | ANAPC1 |
| IL15 | PCDH8 | H2AFY2 | CPNE6 | EMP3 | KCNAB2 | CCK | INTS9 |
| MYO1G | TMIE | OLIG2 | NDST1 | ARAP3 | ASB13 | CCK | MAN2B1 |
| TNFAIP8 | TTN | OLIG2 | CLSTN1 | EMP3 | ASB13 | GABRA1 | PPP1R18 |
| MMP19 | TTN | TMEFF2 | GDA | EMP3 | GALNT13 | GABRA1 | INTS9 |
| IL15 | GABRA1 | PTS | DYNLT3 | MCCC1 | MAGEH1 | CPNE6 | ARHGAP17 |
| LCK | PPP1R1C | SOX6 | TMEM127 | PDPN | WAC | FAM131A | NUP188 |
| CD3G | CACNG3 | PTS | WIPF3 | HOPX | ACTR1A | SYNPR | ARHGAP17 |
| MMP19 | SLC25A32 | EBF1 | OCIAD2 | TLK1 | MXI1 | UGP2 | PTBP1 |
| MMP19 | AIFM2 | TMEFF2 | RBFOX1 | TLK1 | MICU1 | SYNPO | HNRNPAB |
| BATF | SYNPR | PATZ1 | TMEM127 | NPNT | SH3GL2 | SLC6A7 | TTN |
| LY96 | MEIS1 | H2AFY2 | GDA | FABP5 | NALCN | CRTC1 | MIDN |
| BATF | RBP4 | FAM110B | TECPR2 | WIPF3 | KCNAB2 | UGP2 | HNRNPAB |

TABLE 8
Gene Pairs For KIRC Sub-Types

| Solid Tissue | Solid Tissue | | | | | | | | |
|---|---|---|---|---|---|---|---|---|---|
| Normal_1 | Normal_2 | 3_1 | 3_2 | 1_1 | 1_2 | 2_1 | 2_2 | 4_1 | 4_2 |
| PIK3C2G | SIGLEC10 | ADAM12 | FAAH | ATP11A | PPIA | TAZ | POP4 | TIMM8B | ATG2B |
| FXYD4 | COL23A1 | ADAM12 | CCDC130 | TOLLIP | SLC25A39 | TUBGCP6 | TSN | MTX1 | RAD54L2 |
| FXYD4 | NDUFA4L2 | ADAM12 | CRB3 | ATP11A | OAZ1 | TUBGCP6 | STRAP | POP4 | TAF1 |
| CLDN8 | DDB2 | ARL4C | SHMT1 | SPATA18 | MRPS34 | CCDC130 | COPS4 | TIMM8B | ZFHX3 |
| CLDN8 | SEMA5B | CTHRC1 | ACADL | OSBPL1A | SLC25A39 | TUBGCP6 | MMADHC | MRPS34 | UBR5 |
| CLDN8 | STC2 | IL2RA | TMEM171 | ITGA6 | OAZ1 | ZNF692 | COPS4 | POP4 | PRDM2 |
| PIK3C2G | CXXC4 | TRAM2 | PRKAB1 | RAPGEF2 | SLC25A39 | CCDC84 | POP4 | MRPS34 | HERC1 |
| PLA2G4F | STC2 | PLAUR | ACADL | PRUNE2 | OAZ1 | CCDC84 | PIGC | MRPS34 | ARID1B |
| GGT6 | STC2 | ARL4C | IMPA2 | SPATA18 | PSMB3 | TAZ | PIGC | MRPL17 | NEK9 |
| GGT6 | HILPDA | SAP30 | ACADL | SPATA18 | GNG5 | ZNF276 | COPS4 | POP4 | ZFHX3 |
| FAM3B | SPAG4 | ADAMTS12 | TRPM3 | DIP2B | PNKD | ZNF276 | PIGC | GRB2 | MACF1 |
| FAM3B | SAP30 | ARL4C | ACAA2 | BCL2 | TMEM219 | ZNF276 | SPTY2D1 | MTX1 | NEMF |
| FAM3B | TRDMT1 | PODNL1 | C16orf86 | DIP2B | SEC13 | CHKB | LSM11 | ORAI3 | ZFHX3 |
| SLC26A7 | SCARB1 | RUNX1 | PDZK1 | TMCC3 | SEC13 | CCDC130 | POP4 | MTX1 | ZNF445 |
| TMPRSS2 | SCARB1 | ADAMTS12 | FAAH | TMCC3 | PSMB3 | LCAT | GPN3 | LSM4 | ARID1A |
| TMPRSS2 | EGLN3 | CALU | PEBP1 | RIT1 | GTF3A | CHKB | KIAA0391 | TXNDC17 | NR2C2 |
| FXYD4 | BHLHE41 | ADAMTS12 | PTH2R | TMCC3 | GNG5 | GPS2 | HSF2 | CLPP | HERC1 |
| PIK3C2G | CENPP | BCAT1 | ETFDH | ARHGAP42 | PNKD | CCDC130 | MMGT1 | ORAI3 | DICER1 |
| PLA2G4F | SEMA5B | RUNX1 | RIT1 | LYSMD3 | LSM4 | TAZ | USP39 | PRELID1 | ARID1A |
| PLA2G4F | COL23A1 | RUNX1 | TOLLIP | RAVER2 | SLC50A1 | CCDC84 | MMGT1 | MRPL51 | UBR5 |

TABLE 9
Gene Pairs For HNSC Sub-Types

| Solid | Solid | | | | |
|---|---|---|---|---|---|
| Tissue | Tissue | | | | |
| Normal_1 | Normal_2 | Atypical_1 | Atypical_2 | Classical_1 | Classical_2 |
| FAM3D | TGFB1 | ME11 | VEGFC | ASNS | SAMHD1 |
| FAM107A | LOXL2 | ME11 | PDGFC | TMEM116 | CCDC69 |
| CLEC3B | NID2 | FOXRED2 | PRSS23 | SCN9A | APOL3 |
| EMCN | NID2 | ZNF541 | VEGFC | OSGIN1 | SAMHD1 |
| GPD1L | ELF4 | ZNF541 | DACT1 | ARTN | MOB3B |
| FAM3D | TTYH3 | SYCP2 | PODNL1 | SCN9A | CCDC69 |
| CLEC3B | ASPN | MEI1 | FSTL3 | EPCAM | SAMHD1 |
| SH3BGRL2 | TGFB1 | FOXRED2 | USP10 | B4GALNT4 | CCDC69 |
| SH3BGRL2 | TTYH3 | SYNGR3 | FSTL3 | GUI | APOL3 |
| SH3BGRL2 | DNAJC13 | SYCP2 | VEGFC | TMEM116 | ARHGEF10L |
| CLEC3B | PCDH12 | FOXRED2 | FBLIM1 | SCN9A | UBA7 |
| FAM107A | ADAMTS2 | ZNF541 | P4HA3 | CYP4F11 | IL4R |
| FAM3D | TPX2 | SYNGR3 | FBXO44 | TMEM116 | UBA7 |
| GPD1L | MYBL2 | SYNGR3 | PRR5 | PANX2 | TMEM51 |
| NRG2 | NOX4 | CEP70 | PDGFC | ARTN | APOL3 |
| GPD1L | FOXM1 | SYCP2 | F2RL1 | CYP4F11 | RAP1A |
| FAM107A | OLFML2B | ILDR1 | PDGFC | GLI2 | TMEM51 |
| ATP6V0A4 | LOXL2 | C19orf57 | UBTD1 | CYP4F11 | PRDM2 |
| PLIN4 | LOXL2 | FAM83E | PAQR5 | RIT1 | RAP1A |
| NDRG2 | LAMC2 | FAM83E | RUSC2 | OSGIN1 | CASP4 |

| Mesenchymal_1 | Mesenchymal_2 | Basal_1 | Basal_2 |
|---|---|---|---|
| ASPN | RAPGEFL1 | RGS20 | ZDHHC2 |
| POSTN | CD9 | TRPV3 | ZDHHC2 |
| OLFML2B | MAPK13 | TRPV3 | GPRC5B |
| OLFML2B | RAPGEFL1 | HTR7 | GPRC5B |
| TGFB3 | ERBB3 | TRPV3 | PBX1 |
| ASPN | ERBB3 | HTR7 | EPS8 |
| PCOLCE | MAPK13 | RGS20 | GPRC5B |
| ADAMTS2 | SLC9A3R1 | FLRT3 | PTPRS |
| PCOLCE | RAPGEFL1 | GOLGA7B | NTRK2 |
| ASPN | ELF3 | FLRT3 | PBX1 |
| PCOLCE | RAB25 | HTR7 | ZDHHC2 |
| OLFML2B | STAP2 | RGS20 | EPS8 |
| DACT1 | CAMSAP3 | FLRT3 | LTBP3 |
| OLFML3 | STAP2 | SLC6A11 | PBX1 |
| FAP | LLGL2 | SH2D5 | EPS8 |
| GLT8D2 | CAMSAP3 | CDSN | ARHGAP24 |
| OLFML3 | LLGL2 | SLC6A11 | NTRK2 |
| TGFB3 | STAP2 | MOB3B | NTRK2 |
| ADAMTS2 | MAPK13 | TSPAN10 | ARHGAP24 |
| ADAMTS2 | CLDN4 | SH2D5 | TTC28 |

TABLE 10
Gene Pairs For ESCA Sub-Types

| AC_1 | AC_2 | ESCC_1 | ESCC_2 |
|---|---|---|---|
| HNF4A | TFAP2C | TP63 | YKT6 |
| HNF4A | RNF217 | TP63 | BRD2 |
| HNF4A | GPR87 | TP63 | ATG3 |
| MUC13 | BNC1 | ZNF385A | YKT6 |
| MUC13 | SOX15 | S1PR5 | CD68 |
| MUC13 | TP63 | EFS | MRPL1 |
| EPS8L3 | LPAR3 | S1PR5 | PDF |
| EPS8L3 | S1PR5 | S1PR5 | ECM2 |
| EPS8L3 | GPR87 | SOX15 | TIMM8A |
| USH1C | LPAR3 | EFS | ECM2 |
| USH1C | MRPL1 | DSC3 | YKT6 |
| TSPAN8 | MCC | TFAP2C | MCTP2 |
| TSPAN8 | RNF217 | PKP1 | BRD2 |
| TSPAN8 | EFS | EFS | MRPL23 |
| LGALS4 | CALML3 | SOX15 | MCTP2 |
| LGALS4 | TP63 | SNAI2 | TM2D2 |
| TMC5 | SOX15 | PARD6G | MRPL1 |
| GPR35 | S1PR5 | BNC1 | TIMM8A |
| PLEKHA6 | EFS | SNAI2 | MRPL1 |
| PRR15L | EFS | DSC3 | ATG3 |
| VIL1 | LPAR3 | LPAR3 | CD68 |
| VIL1 | S1PR5 | CALML3 | MCTP2 |
| LGALS4 | BNC1 | CALML3 | MRPL23 |
| TMC5 | TFAP2C | CALML3 | TM2D2 |
| TMC5 | MCC | PKP1 | SEC31A |
| HNF1A | PDF | BNC1 | MRPL23 |
| PLEKHA6 | MCC | DSC3 | BRD2 |
| PRR15L | SOX15 | BNC1 | CD68 |
| SEMA4G | GPR87 | FRMD6 | ATG3 |
| USH1C | PARD6G | GPR87 | ECM2 |
| PLEKHA6 | TP63 | SOX15 | IFIT2 |
| PRR15L | TFAP2C | GPR87 | TIMM8A |
| VIL1 | TIMM8A | RNF217 | TM2D2 |
| ICA1 | PARD6G | FSCN1 | SEC31A |
| HNF1A | CD68 | GPR87 | PDF |
| HNF1A | CYB5D1 | LPAR3 | PDF |
| RHPN2 | BNC1 | LPAR3 | CYB5D1 |
| GPR35 | PARD6G | S100A2 | SEC31A |
| GPR35 | TIMM8A | SNAI2 | MRPL18 |
| HNF1B | TIMM8A | FRMD6 | ANGPTL2 |
| SEMA4G | SNAI2 | PKP1 | MRPL18 |
| SLC44A4 | RNF217 | S100A2 | MRPL18 |
| CGN | FRMD6 | PARD6G | IFIT2 |
| RHPN2 | SNAI2 | RHPN2 | SLC44A4 |
| ICA1 | SNAI2 | S100A2 | ANGPTL2 |
| RHPN2 | FRMD6 | RNF217 | IFIT2 |
| SLC44A4 | FRMD6 | GPR35 | VIL1 |
| SLC44A4 | CALML3 | MCC | ANGPTL2 |
| FOXA3 | CHST6 | RNF217 | SIGLEC1 |
| CGN | ZNF385A | SEMA4G | SLC44A4 |

TABLE 11
Gene Pairs For COAD Sub-Types

| Solid | Solid | | | | | | |
|---|---|---|---|---|---|---|---|
| Tissue | Tissue | | | | | | |
| Normal_1 | Normal_2 | CIN_1 | CIN_2 | MSI/CIMP_1 | MSI/CIMP_2 | Invasive_1 | Invasive_2 |
| ABCA8 | URB2 | TNNC2 | CCL5 | ADAMTS2 | SLC39A5 | APOBEC1 | FGFR1 |
| ABCA8 | SLCO4A1 | GDPD5 | TRIM69 | ADAM12 | SGK2 | QPCT | SIRPA |
| ABCA8 | TRIB3 | GDPD5 | ICAM1 | TREM1 | SLC19A3 | QPCT | AQP1 |
| CA7 | FTSJ1 | TTI1 | LHFPL2 | ADAMTS2 | IHH | IL33 | TNS1 |
| CA7 | GTF2IRD1 | SLC5A6 | LGMN | OLR1 | SLC19A3 | QPCT | TNS1 |
| CA7 | KRT80 | MOCS3 | TRIM69 | SLC11A1 | PPP1R14C | COMMD10 | AQP1 |
| SCARA5 | SLC7A5 | TGIF2 | TRIM69 | ADAM12 | PPP1R14C | APOBEC1 | SIRPA |
| SCARA5 | FTSJ1 | CDK5RAP1 | LHFPL2 | SLC11A1 | PLA2G4F | APOBEC1 | CCDC80 |
| SCARA5 | GTF2IRD1 | PIGU | LHFPL2 | HAPLN3 | ABAT | IL33 | SIRPA |
| CLEC3B | KRT80 | TNNC2 | TNFAIP8 | ITGAX | SGK2 | SLC11A2 | AEBP1 |
| CLEC3B | SLCO4A1 | GNG4 | SGMS2 | ICAM1 | SLC39A5 | SMAGP | AEBP1 |
| CLEC3B | TEAD4 | TNNC2 | HPSE | CLEC5A | SLC19A3 | PPA2 | TIMP2 |
| SPIB | URB2 | SLC5A6 | VAPA | NCF2 | SGK2 | RAB32 | AQP1 |
| SPIB | SLCO4A1 | GNG4 | ABHD3 | OSM | RNLS | CYP39A1 | GPR161 |
| SPIB | TEAD4 | SLC35C2 | LGMN | SPP1 | CXCL14 | COMMD10 | TNS1 |
| GLP2R | KRT80 | SLC13A3 | FCGR3A | TREM1 | RNLS | IL33 | EHD2 |
| GLP2R | CLDN1 | GDPD5 | TRIB2 | SLC11A1 | PRRG2 | HSD17B4 | VIM |
| GLP2R | ETV4 | GNG4 | CD163 | C5AR1 | PPP1R14C | SLC11A2 | IGFBP5 |
| TMIGD1 | URB2 | FITM2 | ABHD3 | SPHK1 | PRRG2 | SLC11A2 | TIMP2 |
| TMIGD1 | TEAD4 | SLC13A3 | TAGAP | ITGAX | ABAT | HCN1 | CCDC8 |

TABLE 12
Gene Pairs For BRCA Sub-Types

| Solid Tissue | Solid Tissue | | | | |
|---|---|---|---|---|---|
| Normal_1 | Normal_2 | LumA_1 | LumA_2 | Basal_1 | Basal_2 |
| CD300LG | MMP11 | DEGS2 | PHGDH | FOXC1 | AR |
| TMEM132C | COL10A1 | AGR3 | AIF1L | NEK2 | FOXA1 |
| CA4 | COL10A1 | TMC4 | PHGDH | FAM171A1 | AR |
| ABCA10 | MMP11 | DEGS2 | AIF1L | BCL11A | AGR2 |
| ARHGAP20 | MMP11 | AGR3 | PHGDH | NUSAP1 | MLPH |
| FXYD1 | COL10A1 | ZMYND10 | PSAT1 | CDK1 | FOXA1 |
| PAMR1 | SLC35A2 | FGD3 | IFRD1 | ZWINT | MLPH |
| CD300LG | PAFAH1B3 | MAPT | AIF1L | FOXC1 | MAGI1 |
| TSLP | NEK2 | AGR3 | ID4 | CDK1 | MLPH |
| PAMR1 | PSENEN | DEGS2 | MCCC1 | NUSAP1 | FOXA1 |
| PAMR1 | PYCR1 | ABAT | LPIN1 | FOXC1 | EZH1 |
| CD300LG | TK1 | THSD4 | EGFR | CDCA7 | AR |
| SCARA5 | CENPF | ZMYND10 | CENPW | KCNK5 | AGR2 |
| BTNL9 | SLC50A1 | ZMYND10 | CENPN | NEK2 | AGR2 |
| MAMDC2 | SLC50A1 | FGD3 | TTLL4 | CENPW | SIDT1 |
| ARHGAP20 | TPX2 | FGD3 | LBR | BCL11A | SPDEF |
| MAMDC2 | PYCR1 | ESR1 | CX3CL1 | ORC1 | SIDT1 |
| ARHGAP20 | ZWINT | ABAT | MCCC1 | BCL11A | VIPR1 |
| MAMDC2 | SLC35A2 | ESR1 | EGFR | NEK2 | SPDEF |
| SCARA5 | SLC50A1 | GATA3 | YBX1 | CENPA | SIDT1 |
| LYVE1 | TK1 | NAT1 | LBR | KCNK5 | FBP1 |
| SCARA5 | TIMELESS | SUSD3 | MCCC1 | KCNK5 | THSD4 |
| FXYD1 | NEK2 | KCNJ11 | PSAT1 | CDCA7 | SPDEF |
| CA4 | NEK2 | ABAT | IFRD1 | SKP2 | CMBL |
| LYVE1 | MKI67 | KCNJ11 | DSCC1 | SRSF12 | DNALI1 |
| LYVE1 | LMNB1 | ESR1 | ANO6 | MTHFD1L | CMBL |
| CLEC3B | PAFAH1B3 | FOXA1 | PGRMC1 | CDCA7 | FBP1 |
| BTNL9 | SLC35A2 | MAPT | EGFR | SFT2D2 | REEP5 |
| CLEC3B | TK1 | MLPH | HNRNPD | MTHFD1L | FBP1 |
| CA4 | ASF1B | CA12 | CX3CL1 | PSAT1 | CMBL |
| TSLP | CCNE2 | EVL | KARS | CENPF | GATA3 |
| BTNL9 | PAFAH1B3 | NAT1 | SKP2 | TPX2 | GATA3 |
| TSLP | CENPK | KCNJ11 | PIR | CHODL | DNALI1 |
| C1QTNF9 | CDC25C | SUSD3 | RGMA | SFT2D2 | RHOB |
| ABCA10 | TPX2 | SLC44A4 | KCMF1 | TPX2 | TBC1D9 |
| ABCA10 | ZWINT | NAT1 | IFRD1 | PPP1R14C | THSD4 |
| ASPA | ASF1B | SLC44A4 | LPIN1 | VGLL1 | DNALI1 |
| C1QTNF9 | TAS1R3 | SUSD3 | TTLL4 | VGLL1 | VIPR1 |
| ASPA | DTL | GATA3 | HNRNPD | KRT16 | THSD4 |
| GLYAT | ASF1B | TMC4 | KCMF1 | LMNB1 | TBC1D9 |
| ASPA | CDK1 | CA12 | YBX1 | FAM171A1 | EZH1 |
| CLEC3B | PYCR1 | EVL | HNRNPD | MKI67 | GATA3 |
| C1QTNF9 | CENPA | MAPT | LPIN1 | PPP1R14C | VIPR1 |
| ACVR1C | TPX2 | MLPH | CX3CL1 | NUSAP1 | TBC1D9 |
| GLYAT | DTL | SLC44A4 | TOMM22 | EN1 | TMEM86A |
| ACVR1C | CENPF | MLPH | ORMDL3 | KARS | REEP5 |
| TMEM132C | CDK1 | GATA3 | ARL6IP1 | TPX2 | CA12 |
| ITM2A | UBE2E1 | DNALI1 | RGMA | EN1 | CROT |
| GLYAT | CDK1 | FOXA1 | TRIM29 | UGT8 | CROT |
| TMEM132C | ZWINT | FOXA1 | STAU1 | CDK1 | CA12 |
| LumB_1 | LumB_2 | Normal_1 | Normal_2 | Her2_1 | Her2_2 |
| MCM10 | SFRP1 | CFI | HLTF | MPHOSPH6 | ASB13 |
| CENPA | FOXC1 | LZTS1 | HLTF | GRB7 | IGF1R |
| ESPL1 | SFRP1 | COL17A1 | PEX19 | SIDT1 | IGF1R |
| ESPL1 | CX3CL1 | SERPINF2 | LYSMD1 | MPHOSPH6 | SCARB1 |
| DSCC1 | SFRP1 | COL17A1 | OTUD7B | MPHOSPH6 | SMAD4 |
| CCNE2 | EGFR | LZTS1 | PIGM | PGAP3 | IGF1R |
| CDC25C | TRIM29 | IL3RA | ERI2 | PNMT | ZNF516 |
| CENPK | ID4 | CX3CL1 | ZNF664 | KMO | ASB13 |
| ESPL1 | SLC25A37 | ITM2A | COG2 | PNMT | GREB1 |
| CCNE2 | TRIM29 | PPM1F | OTUD7B | KMO | BCL2 |
| MCM10 | CRYAB | ITM2A | STRBP | PNMT | C1orf226 |
| EME1 | TRIM29 | CFI | COG2 | MFSD2A | RARG |
| DSCC1 | FAM171A1 | CX3CL1 | SDHC | TMEM86A | ASB13 |
| CDC25C | RGMA | NGFR | COG2 | FA2H | C1orf226 |
| MCM10 | FAM171A1 | CX3CL1 | PEX19 | TCAP | NUDT6 |
| ORC1 | FOXC1 | ITM2A | KLHL12 | SPINK8 | RERG |
| WDR76 | EGFR | PPM1F | HLTF | KMO | EZH1 |
| CENPN | FAM171A1 | NGFR | EZH1 | TMEM86A | SCARB1 |
| CENPA | ID4 | CFI | OTUD7B | MFSD2A | SCARB1 |
| NEK2 | SLC25A37 | PPM1F | KLHL12 | SPINK8 | ZNF516 |
| DSCC1 | CRYAB | LZTS1 | RBBP5 | TMEM86A | BCL2 |
| CDC25C | ID4 | COL17A1 | STRBP | ZP2 | EDN3 |
| CCNE2 | CRYAB | PTN | RBBP5 | FGFR4 | STC2 |
| CENPA | RGMA | NGFR | MAGI1 | GRB7 | STC2 |
| NEK2 | GSTP1 | PTN | PEX19 | SPINK8 | GREB1 |
| CDK1 | GSTP1 | PAMR1 | LYSMD1 | MFSD2A | RERG |
| TPX2 | GSTP1 | RHOJ | WDR19 | NUDT8 | C1orf226 |
| CDC25A | FOXC1 | MAMDC2 | LYSMD1 | FA2H | ZNF516 |
| ORC1 | RGMA | RHOJ | ERI2 | FA2H | RERG |
| WDR76 | SLC25A37 | PTN | PIGM | GRB7 | SMAD4 |
| PRIM1 | EGFR | EGFR | GNPAT | SIDT1 | BCL2 |
| WDR76 | TINAGL1 | IL3RA | TADA1 | ZP2 | NUDT6 |
| NEK2 | CX3CL1 | RHOJ | PIGM | SOX11 | RARG |
| RACGAP1 | PNRC1 | PAMR1 | TADA1 | ZP2 | MRGPRX3 |
| DTL | PNRC1 | CHST3 | RBBP5 | FGFR4 | RARG |
| CENPK | ANXA3 | PAMR1 | MBOAT1 | B4GALNT2 | MBOAT1 |
| CENPN | TCF7L1 | PDGFA | PCCB | FGFR4 | EZH1 |
| FANCI | PNRC1 | TINAGL1 | STRBP | TCAP | KIAA0391 |
| CENPN | CHST3 | TRIM29 | GNPAT | DEGS2 | ESR1 |
| DTL | CX3CL1 | SERPINF2 | MBOAT1 | SOX11 | SMAD4 |
| EME1 | ANXA3 | TRIM29 | RRM1 | TCAP | GREB1 |
| PRIM1 | TINAGL1 | PGC | IARS2 | NUDT8 | STC2 |
| PRIM1 | TCF7L1 | PGC | PGRMC1 | CCNE2 | MBOAT1 |
| BRCA1 | TINAGL1 | PGC | HNRNPD | PSMD3 | RPS19 |
| ORC1 | ANXA3 | CADM3 | EPS15 | ABCC2 | NUDT6 |
| DSN1 | PPM1F | EDN3 | NUDT6 | NUDT8 | EZH1 |
| CDC25A | TCF7L1 | TINAGL1 | KLHL12 | SLC44A4 | ESR1 |
| BRCA1 | PDZRN3 | PNRC1 | SDHC | TAS1R3 | PMAIP1 |
| TMEM106C | ZFP36L2 | PDGFA | RRM1 | CDK1 | ESR1 |
| CENPK | BOC | EGFR | RRM1 | ORC1 | PMAIP1 |

TABLE 13
Parameters Used to Train CCN (CCN Subclass Classifiers)

| | final | CCN cross | CCN cross | | | | |
|---|---|---|---|---|---|---|---|
| | general | species | technology | | | | |
| Parameters | CCN | validation | validation | BRCA | COAD | ESCA | HNSC |
| nTopGenes | 25 | 25 | 25 | 20 | 20 | 20 | 20 |
| nTopGenePairs | 70 | 70 | 70 | 50 | 20 | 50 | 20 |
| nRand | 70 | 38 | 70 | 20 | 20 | 20 | 15 |
| nTrees | 2000 | 2000 | 2000 | 2000 | 2000 | 1000 | 2000 |
| stratify | TRUE | TRUE | TRUE | TRUE | TRUE | TRUE | TRUE |
| sampsize | 60 | 25 | 60 | 20 | 24 | 70 | 40 |
| weightedDown_total | 5.00E+05 | 5.00E+05 | 5.00E+05 | 5.00E+05 | 5.00E+05 | 5.00E+05 | 5.00E+05 |
| weightedDown_dThresh | 0.25 | 0.25 | 0.25 | 0.25 | 0.25 | 0.25 | 0.25 |
| transprop_xFact | 1.00E+05 | 1.00E+05 | 1.00E+05 | 1.00E+05 | 1.00E+05 | 1.00E+05 | 1.00E+05 |
| weight_broadClass | NA | NA | NA | 1 | 1 | 5 | 5 |
| quickPairs | TRUE | TRUE | TRUE | FALSE | FALSE | FALSE | FALSE |

| Parameters | KIRC | LGG | UCEC | PAAD | STAD | LUAD | LUSC |
|---|---|---|---|---|---|---|---|
| nTopGenes | 20 | 20 | 10 | 30 | 20 | 20 | 20 |
| nTopGenePairs | 20 | 50 | 20 | 50 | 15 | 25 | 25 |
| nRand | 15 | 15 | 15 | 20 | 55 | 600 | 600 |
| nTrees | 2000 | 2000 | 1000 | 2000 | 1000 | 2000 | 2000 |
| stratify | TRUE | TRUE | TRUE | TRUE | TRUE | TRUE | TRUE |
| sampsize | 70 | 30 | 15 | 30 | 55 | 60 | 27 |
| weightedDown_total | 5.00E+05 | 5.00E+05 | 5.00E+05 | 5.00E+05 | 5.00E+05 | 5.00E+05 | 5.00E+05 |
| weightedDown_dThresh | 0.25 | 0.25 | 0.25 | 0.25 | 0.25 | 0.25 | 0.25 |
| transprop_xFact | 1.00E+05 | 1.00E+05 | 1.00E+05 | 1.00E+05 | 1.00E+05 | 1.00E+05 | 1.00E+05 |
| weight_broadClass | 1 | 15 | 10 | 5 | 10 | 5 | 5 |
| quickPairs | FALSE | FALSE | FALSE | FALSE | FALSE | FALSE | FALSE |

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, cranial implant devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A method of generating a training classifier at least partially using a computer, the method comprising:
generating, by the computer, one or more training data sets, wherein a given training data set comprises gene expression profiles of subjects having a given tumor type;
identifying, by the computer, intersecting genes between the training data sets and one or more query samples to produce one or more intersecting gene sets;
partitioning, by the computer, the intersecting gene sets into training subsets and validation subsets for a given tumor type;
identifying, by the computer, one or more groups of differentially over-expressed genes, differentially under-expressed genes, and/or least differentially expressed genes in the training subsets to produce one or more baseline gene sets;
generating, by the computer, one or more gene-pairs for one or more of the tumor types from the baseline gene sets;
pair-transforming, by the computer, the gene-pairs to produce one or more binarized training data sets;
selecting, by the computer, one or more discriminatory gene-pairs for at least some of the tumor types;
generating, by the computer, one or more random gene-pair profiles through random permutations of the training data sets, which gene-pair profiles lack tumor type annotation; and,
selecting, by the computer, one or more of the gene-pairs as features to produce a random forest classifier, thereby generating the training classifier.

2. The method of claim 1, wherein the query samples comprise cancer cell line (CCL) samples, patient derived xenograft (PDX) samples, and/or genetically engineered mouse model (GEMM) samples.

3. The method of claim 1, wherein the partitioning step comprises randomly sampling the gene expression profiles for the given tumor type.

4. The method of claim 1, comprising evaluating performance of the training classifier using precision-recall curve and area under the precision-recall curve (AUPR).

5. The method of claim 1, comprising repeating one or more steps of generating the training classifier.

6. The method of claim 1, wherein the gene-pairs are selected from genes listed in Table 1.

7. The method of claim 1, comprising adding one or more additional features to produce the random forest classifier.

8. The method of claim 1, comprising evaluating one or more cancer cell line (CCL) expression profiles, patient derived xenograft (PDX) expression profiles, and/or genetically engineered mouse model (GEMM) expression profiles using the training classifier.

9. The method of claim 1, wherein the gene-pairs comprise genes from different species.

10. The method of claim 1, wherein gene expression profiles comprise RNA-seq and/or microarray gene expression profiles.

11. The training classifier generated by the method of claim 1.

12. The method of claim 1, further comprising generating one or more tumor sub-type classifiers.

13. The method of claim 12, wherein the tumor sub-type classifiers comprise one or more gene pairs selected from genes listed in Tables 2-12.

14. A method of evaluating a cancer model at least partially using a computer, the method comprising:
generating, by the computer, one or more training data sets, wherein a given training data set comprises gene expression profiles of subjects having a given tumor type;
identifying, by the computer, intersecting genes between the training data sets and one or more query samples to produce one or more intersecting gene sets;
partitioning, by the computer, the intersecting gene sets into training subsets and validation subsets for a given tumor type;
identifying, by the computer, one or more groups of differentially over-expressed genes, differentially under-expressed genes, and/or least differentially expressed genes in the training subsets to produce one or more baseline gene sets;
generating, by the computer, one or more gene-pairs for one or more of the tumor types from the baseline gene sets;
pair-transforming, by the computer, the gene-pairs to produce one or more binarized training data sets;
selecting, by the computer, one or more discriminatory gene-pairs for at least some of the tumor types;
generating, by the computer, one or more random gene-pair profiles through random permutations of the training data sets, which gene-pair profiles lack tumor type annotation;
selecting, by the computer, one or more of the gene-pairs as features to produce a random forest classifier; and,
evaluating one or more cancer models using the random forest classifier.

15. A system, comprising a controller comprising, or capable of accessing, computer readable media comprising non-transitory computer executable instruction which, when executed by at least electronic processor perform, at least:
generating one or more training data sets, wherein a given training data set comprises gene expression profiles of subjects having a given tumor type;
identifying intersecting genes between the training data sets and one or more query samples to produce one or more intersecting gene sets;
partitioning the intersecting gene sets into training subsets and validation subsets for a given tumor type;
identifying one or more groups of differentially over-expressed genes, differentially under-expressed genes, and/or least differentially expressed genes in the training subsets to produce one or more baseline gene sets;
generating one or more gene-pairs for one or more of the tumor types from the baseline gene sets;
pair-transforming the gene-pairs to produce one or more binarized training data sets;
selecting one or more discriminatory gene-pairs for at least some of the tumor types;
generating one or more random gene-pair profiles through random permutations of the training data sets, which gene-pair profiles lack tumor type annotation; and,
selecting one or more of the gene-pairs as features to produce a random forest classifier, thereby generating the training classifier.

16. The system of claim 15, comprising stratifying sampling when selecting gene-pairs as features to produce the random forest classifier.

17. The system of claim 15, comprising repeating one or more steps of generating the training classifier.

18. The system of claim 15, wherein the gene-pairs are selected from genes listed in Table 1.

19. The system of claim 15, further comprising generating one or more tumor sub-type classifiers.

20. The system of claim 19, wherein the tumor sub-type classifiers comprise one or more gene pairs selected from genes listed in Tables 2-12.
